(19) United States
(12) Patent Application Publication
(10) Pub. No.: US 2014/0066522 A1
(43) Pub. Date: Mar. 6, 2014

(54) EMULSIONS OF PERFLUOROCARBONS

(71) Applicants: Richard Kiral, Costa Mesa, CA (US); Deborah P. Thompson, Durham, NC (US); Gary L. Clauson, Costa Mesa, CA (US)

(72) Inventors: Richard Kiral, Costa Mesa, CA (US); Deborah P. Thompson, Durham, NC (US); Gary L. Clauson, Costa Mesa, CA (US)

(73) Assignee: OXYGEN BIOTHERAPEUTICS, INC., Orange, CA (US)

(21) Appl. No.: 14/011,530

(22) Filed: Aug. 27, 2013

Related U.S. Application Data
(62) Division of application No. 12/761,379, filed on Apr. 15, 2010.
(60) Provisional application No. 61/281,191, filed on Nov. 13, 2009.
(60) Provisional application No. 61/279,359, filed on Oct. 19, 2009.
(60) Provisional application No. 61/214,992, filed on Apr. 29, 2009.
(60) Provisional application No. 61/212,689, filed on Apr. 15, 2009.

(51) Int. Cl. A01N 1/02; A61Q 19/08; A61K 8/06; A61K 9/107; A61K 47/06

(52) U.S. Cl. CPC A01N 1/021; A61K 9/107; A61K 47/06; A61K 8/06; A61Q 19/08
USPC 514/772; 435/1.1

(57) ABSTRACT

The subject application provides for an emulsion comprising an amount of a perfluorocarbon liquid dispersed as particles within a continuous liquid phase, wherein the dispersed particles have a monomodal particle size distribution and uses thereof. The subject application also provides for a method of manufacturing a perfluorocarbon emulsion, a process for preparing a pharmaceutical product containing a PFC emulsion and a process for validating a batch of an emulsion for pharmaceutical use.

This application is a divisional of U.S. Ser. No. 12/761,379, filed Apr. 15, 2010, which claims the benefit of U.S. Provisional Application No. 61/281,191, filed Nov. 13, 2009, U.S. Provisional Application No. 61/279,359, filed Oct. 19, 2009, U.S. Provisional Application No. 61/214,992, filed Apr. 29, 2009 and U.S. Provisional Application No. 61/212,689, filed Apr. 15, 2009, the entire content of each of which is hereby incorporated by reference herein.

Throughout this application various publications, published patent applications, and patents are referenced. The disclosures of these documents in their entireties are hereby incorporated by reference into this application in order to more fully describe the state of the art to which this invention pertains.

### BACKGROUND OF THE INVENTION

Perfluorocarbons (PFCs) are known to be chemically and biologically inert substances which are capable of dissolving very large volumes of gases, including oxygen and carbon dioxide, at concentrations much larger than water, saline and plasma. In addition, PFCs can transport these gases to diffuse across distances. Thus, PFCs can be a convenient means to deliver high levels of oxygen or other therapeutic gases to tissues and organ systems. As a result of their unique properties, PFCs have emerged as leading candidates for gas-transporting components in the treatment of hypoxia secondary to many acute medical situations (Spahn, 1999; U.S. Patent Application Publication No. 2009-0202617).

PFCs that are commonly used in medical research are biologically inert, biostatic liquids at room temperature with densities of about 1.5-2.0 g/mL and high solubilities for oxygen and carbon dioxide. However, neat PFC liquids are unsuitable for injection into the blood stream because their hydrophobicity makes them immiscible in blood. Transportation of neat perfluorocarbon liquid into small blood vessels may cause vascular obstruction and death. Therefore, perfluorocarbons must be dispersed in physiologically acceptable aqueous emulsions for medical uses which require intravascular injection. See, e.g., L. C. Clark, Jr. et al., “Emulsions of Perfluorinated Solvents for Intravascular Gas Transport”, Fed. Proc., 34(6), pp. 1468-77 (1975); K. Yokoyama et al., “A Perfluorochemical Emulsion As An Oxygen Carrier”, Artif. Organs (Ceve), 8(1), pp. 34-40 (1984); and U.S. Pat. Nos. 4,110,474 and 4,187,252.

U.S. Pat. Nos. 5,514,720, 5,684,050, 5,635,539, 5,171,755, 5,407,962 and 5,536,753 disclose various emulsions of highly fluorinated compounds including perfluorocarbons and which are incorporated by reference herein in their entireties.

Perfluorocarbon emulsions are viewed as a promising technology for a wide array of applications (See, e.g., Spiess, 2009; Spahn, 1999; Mason, 1989). However, numerous safety and efficacy issues discussed in the subject application have not previously been identified and resolved to make perfluorocarbon emulsions clinically useful.

### SUMMARY OF THE INVENTION

The subject application provides for an emulsion comprising an amount of a perfluorocarbon liquid dispersed as particles within a continuous liquid phase, wherein the dispersed particles have a monomodal particle size distribution and uses thereof. The subject application provides for a method of manufacturing a perfluorocarbon emulsion comprising: a) mixing an emulsifier and water together; b) adding perfluorocarbon to the mixture of step a); c) mixing the mixture of step b) to form a coarse emulsion; c) obtaining a sample of the coarse emulsion of step c) and determining particle size distribution of the sample; e) if the sample of step d) has a monomodal particle size distribution, then homogenize the coarse emulsion of step c); and f) obtaining the emulsion. The subject application provides for a process for preparing a pharmaceutical product containing a PFC emulsion, the process comprising: a) obtaining a batch of PFC emulsion or coarse emulsion; b)1) determining the particle size distribution of the batch; 2) determining the total amount of residual fluoride present in the batch; or 3) determining the total amount of lysophosphatidylcholine (LPTC) present in the batch; and c) preparing the pharmaceutical product from the batch only if 1) the batch is determined to have a monomodal particle size distribution; 2) the batch is determined to have less than 40 ppm residual fluoride by weight of the emulsion; or 3) the batch is determined to less than 7 g/L lysophosphatidylcholine (LPTC) by weight of the emulsion. The subject application provides for a process for validating a batch of an emulsion for pharmaceutical use, the process comprising: a)1) determining the particle size distribution of a sample of the batch; 2) determining the total amount of residual fluoride in a sample of the batch; or 3) determining the total amount of lysophosphatidylcholine (LPTC) in a sample of the batch; and b) validating the batch for pharmaceutical use only if 1) the sample of the batch has a monomodal particle size distribution; 2) the batch contains less than 40 ppm residual fluoride by weight of the emulsion; or 3) the batch contains less than 7 g/L lysophosphatidylcholine (LPTC) by weight of the emulsion.

### BRIEF DESCRIPTION OF THE DRAWINGS

FIG. 1 shows a production flow chart for manufacturing the claimed emulsion.

FIG. 2 A) shows an unacceptable coarse emulsion percentile size distribution (PSD) after PFC addition; B) shows an unacceptable coarse emulsion PSD after high shear mixing.

FIG. 3 A) shows the PSD of the coarse emulsion of FIG. 2B after homogenization process at 9,000 psig; B) shows the PSD of the coarse emulsion of FIG. 2B after homogenization process at 15,000 psig.

FIG. 4 A) shows the PSD of the coarse emulsion of FIG. 2B after homogenization process at 20,000; B) shows the PSD of the coarse emulsion of FIG. 2B after homogenization process at 25,000 psig.

FIG. 5 A) shows the PSD of an acceptable coarse emulsion. B) shows the PSD of an acceptable coarse emulsion after high pressure homogenization.

FIG. 6 shows the schematic drawing of a typical homogenization set-up.

### DETAILED DESCRIPTION OF THE INVENTION

### Embodiments of the Invention

The subject application provides for an emulsion comprising an amount of a perfluorocarbon liquid dispersed as particles within a continuous liquid phase, wherein the dispersed particles have a monomodal particle size distribution.

In one embodiment, the emulsion contains less than 40 ppm residual fluoride by weight of the emulsion. In another embodiment, residual fluoride is present in the perfluorocarbon emulsion in an amount of less than 40 ppm by weight of the emulsion. In another embodiment, the emulsion contains less than 30 ppm residual fluoride by weight of the emulsion. In another embodiment, the emulsion contains less than 20 ppm residual fluoride by weight of the emulsion. In another embodiment, the emulsion contains 10 ppm-40 ppm residual fluoride by weight of the emulsion. In yet another embodiment, the emulsion contains 20 ppm-30 ppm residual fluoride by weight of the emulsion.

In one embodiment, the emulsion contains less than 7 g/L lysophosphatidylcholine (LPTC or LPC) by weight of the emulsion. In another embodiment, lysophosphatidylcholine (LPTC) is present in the perfluorocarbon emulsion in an amount of less than 7 g/L by weight of the emulsion. In another embodiment, the emulsion contains less than 3 g/L lysophosphatidylcholine (LPTC) by weight of the emulsion. In another embodiment, the emulsion contains less than 2 g/L lysophosphatidylcholine (LPTC) by weight of the emulsion. In another embodiment, the emulsion contains less than 1.5 g/L lysophosphatidylcholine (LPTC) by weight of the emulsion. In another embodiment, the emulsion contains 1.2 g/L-7 g/L lysophosphatidylcholine (LPTC) by weight of the emulsion. In another embodiment, the emulsion contains 2 g/L-6 g/L lysophosphatidylcholine (LPTC) by weight of the emulsion. In another embodiment, the emulsion contains 3 g/L-5 g/L lysophosphatidylcholine (LPTC) by weight of the emulsion.

In an embodiment, 90% or more of the total amount by volume of the dispersed particles have a size of less than 700 nanometers (nm). In another embodiment, 90% or more of the total amount by volume of the dispersed particles have a size of less than 600 nanometers (nm).

In one embodiment, 50% or more of the total amount by volume of the dispersed particles have a size of less than 400 nanometers (nm). In another embodiment, 50% or more of the total amount by volume of the dispersed particles have a size of less than 300-350 nanometers (nm). In another embodiment, 50% or more of the total amount by volume of the dispersed particles have a size of less than 200-300 nanometers (nm). In another embodiment, 99% or more of the total amount by volume of the dispersed particles have a size of less than 1 microns (μm).

In one embodiment, the D(0.9) of the dispersed particles is about 700 nanometers (nm). In another embodiment, the D(0.9) of the dispersed particles is about 600 nanometers (nm). In another embodiment, the D(0.5) of the dispersed particles is about 150-400 nanometers (nm). In another embodiment, the D(0.5) of the dispersed particles is about 200-330 nanometers (nm). In another embodiment, the D(0.99) of the dispersed particles is about 1 micron (μm). In yet another embodiment, the mean size of the dispersed particles is about 200-400 nm.

In one embodiment, the mean diameter of the dispersed particles is about 0.20-0.25 μm. In another embodiment, the mean diameter of the dispersed particles is about 0.20 μm. In yet another embodiment, the median size of the dispersed particles is about 180-300 nm.

In one embodiment, the perfluorocarbon is perfluoro(tert-butylcyclohexane), perfluorodecalin, perfluoroisopropyldecalin, perfluoro-tripropylamine, perfluorotributylamine, perfluoro-methylcyclohexylpiperidine, perfluoro-octylbromide, perfluoro-decylbromide, perfluoro-dichlorooctane, perfluorohexane, dodecafluoropentane, or a mixture thereof.

In one embodiment, the perfluorocarbon contains less than 5 ppm residual conjugated olefin by weight of the perfluorocarbon. In another embodiment, residual conjugated olefin is present in the perfluorocarbon in an amount of less than 5 ppm by weight of the perfluorocarbon. In another embodiment, the perfluorocarbon contains less than 3 ppm residual conjugated olefin by weight of the perfluorocarbon. In another embodiment, the perfluorocarbon contains less than 1 ppm residual conjugated olefin by weight of the perfluorocarbon.

In one embodiment, the perfluorocarbon contains less than less than 1 ppm residual fluoride by weight of the perfluorocarbon. In another embodiment, residual fluoride is present in the perfluorocarbon in an amount of less than 1 ppm by weight of the perfluorocarbon. In another embodiment, the perfluorocarbon contains less than less than 0.7 ppm residual fluoride by weight of the perfluorocarbon.

In one embodiment, the perfluorocarbon contains less than 20 ppm residual organic hydrogen by weight of the perfluorocarbon. In another embodiment, residual organic hydrogen is present in the perfluorocarbon in an amount of less than 20 ppm by weight of the perfluorocarbon. In one embodiment, the perfluorocarbon contains less than 10 ppm residual organic hydrogen by weight of the perfluorocarbon. In another embodiment, the perfluorocarbon contains less than 5 ppm residual organic hydrogen by weight of the perfluorocarbon.

In one embodiment, the emulsion comprises 20-80% w/v perfluorocarbon. In another embodiment, the emulsion comprises 60% w/v perfluorocarbon.

In one embodiment, the emulsion further comprises an emulsifier. In another embodiment, the emulsion comprises 1-10% w/v emulsifier. In another embodiment, the emulsion comprises 2.5-4.5% w/v emulsifier. In another embodiment, the emulsifier is a surfactant. In yet another embodiment, the surfactant is egg yolk phospholipid.

In one embodiment, the emulsion comprises 40-80% w/v water. In another embodiment, the emulsion comprises 50-70% w/v water. In yet another embodiment, the water is Water for Injection.

In one embodiment, the emulsion further comprises an aqueous medium. In another embodiment, the aqueous medium is isotonic. In another embodiment, the aqueous medium is buffered to a pH of 6.8-7.4. In yet another embodiment, the emulsion further comprises Vitamin E.

The subject application also provides for a method of treating sickle cell disease, decompression sickness, air embolism or carbon monoxide poisoning in a subject suffering therefrom comprising administering to the subject the emulsion described herein effective to treat the subject's sickle cell disease, decompression sickness, air embolism or carbon monoxide poisoning. In one embodiment, the emulsion is administered intravenously (IV) or intrathecally.

The subject application also provides for a method of preserving an organ prior to transplant comprising contacting the organ with the emulsion described herein effective to increase the organ's survival time. In one embodiment, the organ is perfused with the emulsion.

The subject application also provides for a method of treating a wound, a burn injury, acne or rosacea in a subject suffering therefrom comprising topically administering to the skin of the subject the emulsion described herein effective to treat the subject's wound, burn injury, acne or rosacea.

The subject application also provides for a method of increasing the firmness of the skin or reducing the appearance of fine lines, wrinkles or scars in a subject comprising topically administering to the skin of the subject the emulsion described herein effective to increase the firmness of the subject's skin or reduce the appearance of fine lines, wrinkles or scars on the subject's skin.

The subject application also provides for a method of manufacturing a perfluorocarbon emulsion comprising the steps: a) mixing an emulsifier and aqueous medium together; b) adding perfluorocarbon to the mixture of step a); c) mixing the mixture of step b) to form a coarse emulsion; d) obtaining a sample of the coarse emulsion of step c) and determining particle size distribution of the sample; e) if the sample of step d) has a monomodal particle size distribution, then homogenizing the coarse emulsion of step c); and f) obtaining the emulsion.

In one embodiment, in step a) the emulsifier and aqueous medium are mixed together at between 2,000-7,000 rpm.

In one embodiment, in step c) the mixture of step b) is mixed at above 8,000 rpm.

In one embodiment, in step e) the coarse emulsion of step c) is homogenized under high pressure.

In one embodiment, in step d) the particle size distribution is determined using a laser light scattering particle-size distribution analyzer. In another embodiment, in step e) the mixture of step c) is homogenized only if the median particle size of the sample of step d) is less than 20 μm. In another embodiment, in step e) the mixture of step c) is homogenized only if the mixture of step c) has a pH of 6.8-7.4. In another embodiment, in step e) the coarse emulsion is homogenized at or above 7,000 psi. In yet another embodiment, in step f) the emulsion is obtained after a predetermined amount of time. This predetermined amount of time can be the emulsification time which is dependent on batch size and flow rate through the homogenizer. The emulsification time can be determined from a continuous flow calculation and calculated using the calculation disclosed in Leviton and Pallansch. (Leviton, 1959)

The subject application also provides for a process for preparing a pharmaceutical product containing a PFC emulsion, having a monomodal particle size distribution, comprising: a) obtaining a batch of perfluorocarbon emulsion or coarse emulsion; b) determining the particle size distribution of the batch; and c) preparing the pharmaceutical product from the batch only if the batch is determined to have a monomodal particle size distribution.

In one embodiment, in step b) the particle size distribution is determined using a laser light scattering particle-size distribution analyzer.

The subject application also provides for a process for preparing a pharmaceutical product containing a PFC emulsion containing less than 40 ppm residual fluoride by weight of the emulsion, comprising: a) obtaining a batch of perfluorocarbon emulsion or coarse emulsion; b) determining the total amount of residual fluoride present in the batch; and c) preparing the pharmaceutical product from the batch only if the batch is determined to have less than 40 ppm residual fluoride by weight of the emulsion.

The subject application also provides for a process for preparing a pharmaceutical product containing a PFC emulsion less than 7 g/L lysophosphatidylcholine (LPTC), comprising: a) obtaining a batch of perfluorocarbon emulsion or coarse emulsion; b) determining the total amount of lysophosphatidylcholine (LPTC) present in the batch; and c) preparing the pharmaceutical product from the batch only if the batch is determined to have less than 7 g/L lysophosphatidylcholine (LPTC) by weight of the emulsion.

The subject application also provides for a process for validating a batch of an emulsion for pharmaceutical use comprising: a) determining the particle size distribution of a sample of the batch; and b) validating the batch for pharmaceutical use only if the sample of the batch has a monomodal particle size distribution.

In one embodiment, in step a) the particle size distribution is determined using a laser light scattering particle-size distribution analyzer.

The subject application also provides for a process for validating a batch of a emulsion for pharmaceutical use comprising: a) determining the total amount of residual fluoride in a sample of the batch; and b) validating the batch for pharmaceutical use only if the sample of the batch contains less than 40 ppm residual fluoride by weight of the emulsion.

The subject application also provides for a process for validating a batch of a emulsion for pharmaceutical use comprising: a) determining the total amount of lysophosphatidylcholine (LPTC) in a sample of the batch; and b) validating the batch for pharmaceutical use only if the sample of the batch contains less than 7 g/L lysophosphatidylcholine (LPTC) by weight of the emulsion.

In one embodiment, in step a) the sample of the batch has been subjected to stability testing.

All combinations of the various elements described herein are within the scope of the invention.

The biochemistry of wound healing and strategies for wound treatment is described Chin et al., (2007) “Biochemistry of Wound Healing in Wound Care Practice” Wound Care Practice, 2^{nd} ed., Best Publishing, AZ., which is hereby incorporated by reference. Acne treatments are described in section 10, chapter 116, pp 811-813 of The Merck Manual, 17^{th} Edition (1999), Merck Research Laboratories, Whitehouse Station, N.J., U.S.A. which is hereby incorporated by reference. Sickle cell disease treatments are described in section 11, chapter 127, pp 878-883 of The Merck Manual, 17^{th} Edition (1999), Merck Research Laboratories, Whitehouse Station, N.J., U.S.A. which is hereby incorporated by reference.

### TERMS

As used herein, and unless stated otherwise, each of the following terms shall have the definition set forth below.

“About” in the context of a numerical value or range means±10% of the numerical value or range recited or claimed.

“Accelerates healing” as used herein means an increased rate of tissue repair and healing as compared to the rate of tissue repair and healing in an untreated control subject.

“Administering to the subject” means the giving of, dispensing of, or application of medicines, drugs, or remedies to a subject to relieve or cure a pathological condition. Topical administration is one way of administering the instant compounds and compositions to the subject. The administering can also be performed, for example, intravenously or intra-arterially.

“Ameliorating” a condition or state as used herein shall mean to lessen the symptoms of that condition or state. “Ameliorate” with regard to skin comedones, pustules or papule is to reduce the discomfort caused by comedones, pustules or papules and/or to reduce their appearance and/or physical dimensions.

“Antibacterial agent” means a bactericidal compound such as silver nitrate solution, mafenide acetate, or silver sulfadiazine, or an antibiotic. According to the present invention, antibacterial agents can be present in “Curpon™” products. “Cupron™” products utilize the qualities of copper and binds copper to textile fibers, allowing for the production of woven, knitted and non-woven fabrics containing copper-impregnated fibers with the antimicrobial protection against microorganisms such as bacteria and fungi.

“Biologically active agent” means a substance which has a beneficial effect on living matters.

“Burn wound” means a wound resulting from a burn injury, which is a first, second or third degree injury caused by thermal heat, radiation, electric or chemical heat, for example as described at page 2434, section 20, chapter 276, of The Merck Manual, 17^{th} Edition (1999), Merck Research Laboratories, Whitehouse Station, N.J., U.S.A.

“Carbon monoxide poisoning” or “CO poisoning” means the poisoning of a subject resulting from exposure to carbon monoxide. Toxicity of carbon monoxide can vary with the length of exposure, concentration of CO that the subject was exposed to, respiratory and circulatory rates. Symptoms of carbon monoxide poisoning can vary with the percent carboxyhemoglobin present in the blood and can include headache, vertigo, dyspnea, confusion, dilated pupils, convulsions and coma (some of which result from injury to the brain). The standard treatment for CO poisoning is the administration of 100% oxygen by breathing mask (The Merck Manual, 1999; Prockop, 2007).

“Central Nervous System” or “CNS” shall mean the brain and spinal cord of a subject.

“Closed head” injury or “non-penetrating” injury is an injury within the brain where skull penetration has not occurred.

“Effective” as in an amount effective to achieve an and means the quantity of a component that is sufficient to yield a desired therapeutic response with a reasonable benefit/risk ratio when used in the manner of this disclosure. For example, an amount effective to promote wound healing without causing undue adverse side effects. The specific effective amount will vary with such factors as the particular condition being treated, the physical condition of the patient, the type of mammal being treated, the duration of the treatment, the nature of concurrent therapy (if any), and the specific formulations employed and the structure of the compounds or its derivatives.

“Emulsifier” shall mean a substance which stabilizes an emulsion.

“Emulsion” shall mean a mixture of two immiscible liquids. Emulsions are colloids wherein both phases of the colloid (i.e., the dispersed phase and the continuous phase) are liquids and one liquid (the dispersed phase) is dispersed in the other liquid (the continuous phase). The dispersed phase liquid can be, as is often with PFC's, referred to as taking the form of “particles” suspended in the continuous phase liquid. Each use of the term “particle” or “particles” herein is intended to apply to liquid PFC microspheres or droplets in the continuous liquid phase and microbubbles (which make the emulsion in such state a colloidal suspension). In one embodiment of this invention, the emulsion is a perfluorocarbon emulsion and the two immiscible liquids of the perfluorocarbon emulsion are perfluoro(tert-butylcyclohexane) and egg-yolk phospholipid. “Particles” as used herein can also mean microbubbles of a substance in the gaseous phase, e.g., a PFC vapor in the form of a microbubble.

“D(0.5)” is the particle size; in microns, below which 50% by volume distribution of the population is found. “D(0.9)” is the particle size, in microns, below which 90% by volume distribution of the population is found.

“Decompression sickness” or “DCS” means the disorder resulting from reduction of surrounding pressure (e.g., during ascent from a dive, exit from a caisson or hyperbaric chamber, or ascent to altitude), attributed to formation of bubbles from dissolved gas in blood or tissues, and usually characterized by pain and/or neurologic manifestations (The Merck Manual, 1999).

“Fraction of Inspired Oxygen” or “FiO₂” is the amount of oxygen in the air delivered to a subject. The FiO₂ is expressed as a number from 0 (0%) to 1 (100%). The FiO₂ of normal room air is 0.21 (21%), i.e., 21% of the normal room air is oxygen.

As used herein, a composition that is “free” of a chemical entity means that the composition contains, if at all, an amount of the chemical entity which cannot be avoided following an affirmative act intended to separate the chemical entity and the composition.

“Glasgow Coma Scale” or “GCS” shall mean the neurological scale used in determining Best Eye Response, Best Verbal Response, Best Motor Response (see Teasdale G., Jennett B., LANCET (ii) 81-83, 1974). It is a widely used scoring system for quantifying level of consciousness following traumatic brain injury.

“Impaired oxygenation” shall mean, with regard to a tissue or cell, an oxygenation level of the tissue below that which exists in the same tissue or cell under normal physiological conditions.

‘Infection’ as used in respect to Propionibacterium acnes means a detrimental colonization of the (host) subject by the Propionibacterium acnes causing an inflammation response in the subject.

“Ischemic pain” shall mean pain or discomfort caused by localized ischemia in subjects with sickle cell disease.

“Monomodal particle size distribution” shall mean a collection of particles (e.g., liquid microspheres, liquid droplets, powders, granules, beads, crystals, pellets, etc.) which have a single clearly discernable maximum on a particle size distribution curve (weight percent or intensity on the ordinate or Y-axis, and particle size on the abscissa or X-axis). A monomodal particle size distribution is distinct from a bimodal particle size distribution which refers to a collection of particles having two clearly discernable maxima on a particle size distribution curve. A monomodal particle size distribution is also distinct from a multimodal particle size distribution which refers to a collection of particles having three or more clearly discernable maxima on a particle size distribution curve.

“Oxygen tension” or “tissue oxygen tension” is the directly measured local partial pressure of oxygen in a specific tissue.

“Oxygenated perfluorocarbon” is a perfluorocarbon which is carrying oxygen at, for example, saturation or sub-saturation levels.

“Peripheral resistance” shall mean peripheral vascular resistance of the systemic circulation.

“Pharmaceutically acceptable carrier” refers to a carrier or excipient that is suitable for use with humans and/or animals without undue adverse side effects (such as toxicity, irritation, and allergic response) commensurate with a reasonable benefit/risk ratio. It can be a pharmaceutically acceptable solvent, suspending agent or vehicle, for delivering the instant compounds to the subject. The carrier may be liquid or solid and is selected with the planned manner of administration in mind.

“Pharmaceutically active compound” means the compound or compounds that are the active pharmaceutical ingredients in a pharmaceutical formulation. “Active pharmaceutical ingredient” or “API” is defined by U.S. Food and Drug Administration as any substance or mixture of substances intended to be used in the manufacture of a drug product and that, when used in the production of a drug, becomes an active ingredient in the drug product. Such substances are intended to furnish pharmacological activity or other direct effect in the diagnosis, cure, mitigation, treatment or prevention of disease or to affect the structure and function of the body.

“Primary” and “secondary” are classifications for the injury processes that occur in brain injury. In TBI, primary injury occurs during the initial insult, and results from displacement of the physical structures of the brain. Secondary injury occurs gradually and may involve an array of cellular processes. Secondary injury, which is not caused by initial mechanical damage, can result from the primary injury or be independent of it. Therefore, “primary ischemia” is the lack to blood flow (resulting in restriction in oxygen supply) resulting directly from the initial injury to the brain while “secondary ischemia” is the lack to blood flow (resulting in restriction in oxygen supply) resulting from the process initiated by the initial injury, e.g., from complications of the initial injury, and can involve tissues that were unharmed in the primary injury. The primary and secondary classification of TBI is discussed in detail by Silver, J., et al. (2005) “Neural Pathology” Textbook Of Traumatic Brain Injury. Washington, D.C.: American Psychiatric Association. Chap. 2, pp. 27-33.

“Promotes alleviation of pain” means a decrease in the subject's experience of pain resulting from a wound, an injury, e.g., a burn injury or other pathological conditions.

“Sex organ” or “sexual organ” means any of the anatomical parts of the body which are involved in sexual reproduction and/or gratification and constitute the reproductive system in a complex organism. In a preferred embodiment of this invention, the sex organ is the genitalia of the subject. As used herein, the “genitalia” refer to the externally visible sex organs: in males the penis, in females the clitoris and vulva.

“Sickle Cell Disease” is a chronic hemoglobinopathy caused by homozygous inheritance of Hb S.

“Stability testing” refers to tests conducted at specific time intervals and various environmental conditions (e.g., temperature and humidity) to see if and to what extent a drug product degrades over its designated shelf life time. The specific conditions and time of the tests are such that they accelerate the conditions the drug product is expected to encounter over its shelf life. For example, detailed requirements of stability testing for finished pharmaceuticals are codified in 21 C.F.R §211.166, the entire content of which is hereby incorporated by reference.

“Topical administration” of a composition as used herein shall mean application of the composition to the skin or mucous membranes of a subject. In an embodiment, topical administration of a composition is application of the composition to the epidermis of a subject.

“Traumatic Brain Injury” or “TBI” shall mean central nervous system injury, i.e. CNS neuronal, axonal, glial and/or vascular destruction, from an impact. Such impacts include blunt impacts, bullet injury or blast injury.

“Vaso-occlusive crisis” shall mean the clinically recognized condition resulting from sickle-shaped red blood cells obstructing capillaries and restricting blood flow to tissues and/or organs, resulting in, inter alia, ischemia and pain.

“w/v” designates a weight/volume ratio typically used to characterize biological solutions. A 1% w/v solution has 1 g of solute dissolved in a final volume of 100 mL of solution.

### PFC Emulsion Characteristics

Since PFC liquids are not miscible with aqueous systems, including blood and other body fluids, they should be formulated as a physiologically compatible emulsion before it can be administered intravenously.

A number of considerations should be taken into account when formulating a PFC emulsion for injection into the blood stream, including but not limited to, impurities present in the emulsion, emulsion particle size, emulsion particle size distribution and emulsion stability. The ideal PFC emulsion should have the following features regardless of the PFC used in the emulsion.

### Limited Impurities Present in the PFC Emulsion

The ideal PFC emulsion should have minimal levels of impurities. Specifically, the ideal PFC emulsion should have the following characteristics:
1. The perfluorocarbon emulsion contains less than 40 ppm residual fluoride by weight of the emulsion, preferably, less than 20 ppm residual fluoride by weight of the emulsion;
2. The perfluorocarbon emulsion contains less than 7 g/L lysophosphatidylcholine (LPTC), which has been implicated as a potent inflammatory lipid associated with diabetic retinopathy, atherogenesis and neurodegeneration.
3. The perfluorocarbon emulsion contains less than 5 ppm residual conjugated olefin by weight of the perfluorocarbon, preferably less than 1 ppm residual conjugated olefin by weight of the perfluorocarbon;
4. The perfluorocarbon emulsion contains less than 1 ppm, preferably, less than 0.7 ppm residual fluoride by weight of the perfluorocarbon;
5. The perfluorocarbon emulsion contains less than 20 ppm residual organic hydrogen by weight of the perfluorocarbon, preferably less than 5 ppm residual organic hydrogen by weight of the perfluorocarbon.

### Small Particle Size

Very small particle size is a desired trait for a PFC emulsion indicated for injection into the blood stream. It has been shown that size is a major factor determining clearance rate of particles from the circulation, the site of primary clearance and the degree if any of complement activation.

PFCs are not metabolized and are not soluble in water or lipids. Therefore, they are not excreted in urine or feces, but are exhaled by the lungs as the route of elimination. The rate of clearance of PFC emulsions from the blood compartment after intravenous injection has been shown to be dose-dependent and influenced by the emulsion composition. The predominant means of removal from the blood stream is through phagocytosis of emulsion particles by macrophages of the reticuloendothelial system (RES), i.e., largely by fixed macrophages in the spleen and liver.

Particle size distribution is a major determinant of particle clearance by the mononuclear phagocytic system and the potential for concomitant activation of resident macrophages. It is also a major cause of adverse effects. Small particle size would allow particles to evade the RES and remain in the vasculature longer with fewer side effects.

Particle size also correlates directly with emulsion side effects. The distribution of larger particles is associated with more side effects: even if the mean particle size in the emulsion is <0.3 microns, the presence of larger particles increases the chance of an adverse effect.

Studies with various liposomal formulations have suggested that particles ≧0.3 μm in diameter are readily opsonized with complement and cleared more rapidly from the circulation than particles ≦0.2 μm in diameter. Large particles appear to be cleared by the spleen, whereas small particles are cleared predominantly by the liver.

### Monomodal Particle Size Distribution

During the manufacturing of the PFC emulsion, specifically, after the high-speed mixing step and prior to the homogenization step in the manufacturing process, a laser light scattering particle-size distribution analyzer can be used to analyze the particle size distribution of the coarse emulsion. Under the laser light scattering particle-size distribution analyzer, the particles can have a monomodal, a biomodal or a multimodal particle size distribution.

It was surprisingly found by the inventors that only the coarse emulsions which have a monomodal particle size distribution during this intermediate step result in a final emulsion with monomodal particle size distribution. That is, if a second peak is not removed at this stage in the manufacturing process, it remains in the final emulsion. Therefore, the coarse emulsion should only be moved from the high-speed mixer to homogenizer when the coarse emulsion achieved a monomodal particle size distribution under the laser light scattering particle-size distribution analyzer.

### Immunoactivity

The ideal emulsion should not be immunoactive.

A number of early PFC emulsion formulations (e.g., Fluosol DA from Green Cross Corporation, Japan and Perftoran from Perftoran, Russia) have been found to be immunoactive. The surfactant used in these PFC emulsions (Pluronic F68 and Proxanol-268) have been found to activate alternative complement pathway of the immune system.

### High PFC Emulsion Stability

The ideal emulsion should continue to meet all of the stability acceptance specifications during its intended shelf life. The particle size and particle size distribution differ from other specifications because they will change as the emulsion ages. This growth is inevitable because the emulsion, by definition, is thermodynamically unstable. Even a good emulsion will exhibit some growth in particle size during its intended shelf life, whether by Ostwald ripening, coalescence, flocculation, or sedimentation. However, if the emulsion is properly formulated and the manufacturing process is optimized, the particle size growth rate should be reasonably small, the median size should remain in the 200-400 nm range, and the particle size distribution should remain reasonably narrow.

The known PFC emulsions have numerous stability problems. (Fluosol DA (20%): P-F68 is very unstable and the emulsion needs to be stored frozen; Perftoran: stable only 8 hrs post reconstitution; Oxygent™: Degradation products of arachidonic acid may cause flu-like reactions; OxyFluor®: Can be stored without refrigeration for one year only). In comparison with these PFC emulsions, the PFC emulsion disclosed herein is highly stable.

### Additional PFC Emulsion Features

Other considerations to take into account in formulating a PFC emulsion include:
1. the emulsion's effect on development of thrombocytopenia: thrombocytopenia is a disorder in which there is an insufficient number of platelets in the blood;
2. the emulsion's effect on inhibition of platelet aggregation: a number of existing PFC emulsions have been found to inhibit platelet aggregation, which keeps a trauma patient from being predisposed to formation of life-threatening clot, but may also increase risk of intracranial bleed;
3. the emulsion's effect on inhibition of PMN adherence to endothelial cells: neutrophil (PMN) adherence to endothelia cells is thought to be an early event in the sequence resulting in injury to vascular endothelium.
4. the emulsion's effect on activation of macrophages: activated macrophages have increased phagocytic activity, particular with respect to Listeria and Salmonella species. However, activated macrophages can also stimulate production of damaging inflammatory cytokines. For example, exposure of stimulated human alveolar macrophages to Oxygent™ in vitro decreases cytokine production, suggesting that Oxygent™, and likewise Oxycyte®, may have anti-inflammatory activity.
5. the emulsion's effect on immunocompetence, platelet function, and platelet survival: a preferred PFC emulsion do not affect immunocompetence and platelet function of the subject, and should not shorten platelet survival in the subject.

### Perfluoro(Tert-Butylcyclohexane)

PFC molecules are generally accepted to be biologically inert, owing to their extensive halogenation, which creates an electron configuration that is resistant to metabolic degradation. Therefore, traditional forms of toxicity stemming from formation of reactive metabolites or from direct interaction of the PFC with bio-macromolecules have not been an issue for this class of compounds. Similarly, no genetic toxicity has been identified for PFCs. However, PFC dose required for oxygen delivery applications is typically in the range of 2-3 grams per kilogram body weight, which is substantially higher than that of conventional drug products. Thus, sufficient oxygen delivery via intravenous injection of PFCs could entails intravenous delivery of a relatively large quantity of a particulate suspension. As such, the PFC's effect on tissue morphology is an important factor to consider in its selection for this use.

The proper choice of perfluorocarbon should provide the necessary efficacy with proper safety profile. In addition to being safe and effective for its intended use, the perfluorocarbon should also be able to be economically incorporated into stable product formulations. To meet these goals the perfluorocarbon should meet most, preferably all, of the following criteria:
1. The perfluorocarbon should be capable of dissolving and releasing large quantities of gases, especially the blood gases oxygen and carbon dioxide.
2. The perfluorocarbon is preferably composed of only carbon and fluorine.
3. The perfluorocarbon is preferably a single chemical entity with few isomeric and non-isomeric impurities. Residual impurities such as conjugated olefins, organic hydrides, and fluoride should be kept at a ppm level.
4. The perfluorocarbon should be chemically non-reactive and thermally stable at temperatures up to and including those used in typical steam sterilization processes.
5. The perfluorocarbon should be metabolically inert.
6. The perfluorocarbon should be able to be formulated into a stable emulsion of sub-micron sized droplets that can be stored for an extended period of time without significant droplet growth due to coalescence or diffusion-controlled mechanisms. Preferably, the formulation, contains only a single perfluorocarbon.
7. The perfluorocarbon should possess an acceptable safety profile and be devoid of toxicity.
8. In the emulsified form, the perfluorocarbon should have an appreciable residence time in the blood and an acceptable time frame for elimination from the major reticuloendothelial organs of the body.

Ideally, the PFC selected would also have two desired features: rapid RES clearance and minimal potential to cause hyperinflation.

The rate of PFC clearance from and recovery of normal RES histomorphology is positively correlated with the relative lipophilicity of the PFC and, secondarily, to the vapor pressure of the PFC. Although phagocytosis of PFC emulsion particles by RES macrophages is not deleterious to the primary organ of uptake, there are clinical consequences that stem from this process. The best characterized is the flu-like symptoms commonly observed in clinical studies of PFC emulsion products. Therefore, rapid RES clearance is a desired trait for a PFC selected for use in an intravenous emulsion.

Some PFCs in known formulations were selected in part based on their relatively short retention time in the RES. Two such PFCs are perfluorodecalin (PFD), the main constituent of Fluosol DA by the Green Cross Corp. of Japan, which was the first blood substitute to be approved by the FDA, and perfluorooctyl bromide (PFOB), the main component of Oxygent™, a blood substitute by Alliance Pharmaceutical Corp. of San Diego, Calif. PFD and PFOB have vapor pressures of approximately 13 and 10 torr, respectively.

The bias towards selecting PFCs with shorter RES retention times has been tempered over the years by the realization that pulmonary expiration of PFCs is not a benign process. A phenomenon dubbed “pulmonary hyperinflation” was first documented in rabbits. This condition is characterized by a failure of the lungs to collapse to their normal “resting volume”. In rabbits that were treated with single doses of certain PFC emulsions, lungs not only failed to collapse to their resting volume, but also appeared to expand beyond their normal functional residual capacity (i.e., hyperinflates). In its extreme form, respiratory dynamics are affected and gas exchange is compromised, and the condition can be life-threatening. The single most important determinant of the propensity of different PFCs to induce hyperinflation of the lungs is the rate of migration of the PFC into the airspace, which is dependent largely on vapor pressure and secondarily on lipophilicity.

The difficulty in choosing a PFC with optimal properties is that the two most desired features, i.e., rapid RES clearance and minimal potential to cause hyperinflation, are counter-opposing. Selection of a candidate with low vapor pressure that has little or no potential to elicit hyperinflation would result in an unacceptably long RES half-life. While it could be effectively argued that this slower RES clearance is not an important safety concern, persistent organmegaly and associated histopathology could be considered unacceptable from a regulatory standpoint.

Perfluoro(tert-butylcyclohexane) at both 50% and 20% w/v concentrations has been tested in controlled, single-dose Good Laboratory Practice (GLP) toxicity studies in rats and monkeys. In comparison with other PFCs, the degree of hyperinflation seen with perfluoro(tert-butylcyclohexane) was significantly less than that seen in monkeys treated with PFOB, and in previous unpublished studies in rabbits with perfluorodecalin. Absorption of perfluoro(tert-butylcyclohexane) in the body was generally comparable to what has been reported for other PFCs. However, persistence in liver and spleen was somewhat longer than what has been reported for PFOB. Nevertheless, perfluoro(tert-butylcyclohexane) represents a better balance between persistence and the tendency to produce hyperinflated, non-collapsible lungs than what is seen with PFOB and perfluorodecalin.

In addition, in comparison with other perfluorocarbons tested as oxygen carriers, perfluoro(tert-butylcyclohexane) appears on the basis of animal studies to have a better safety profile, and does not contain bromine or chlorine and thus does not pose the risk of ozone depletion. Further, biomedical grade compound can be produced in mass quantities.

Based on the foregoing, the perfluoro(tert-butylcyclohexane) disclosed herein has an optimal balance of properties. Its RES half-life is somewhat longer than that of the benchmark perfluorocarbon, PFOB, but it has a correspondingly lesser propensity to cause pulmonary hyperinflation. Overall, Perfluoro(tert-butylcyclohexane) appears to be a good candidate for use in an intravenous PFC emulsion.

Perfluoro(tert-butylcyclohexane) (C₁₀F₂₀) is available, for example, from Oxygen Biotherapeutics Inc., Costa Mesa, Calif.

Oxycyte® is a perfluorocarbon emulsion oxygen carrier. The active ingredient in Oxycyte®, perfluoro(tert-butylcyclohexane) (C₁₀F₂₀, MW=500.08), also known as F-tert-butylcyclohexane or FtBu, is a saturated alicyclic PFC. Perfluoro(tert-butylcyclohexane) is a colorless, completely inert, non-water soluble, non-lipophilic molecule, which is twice as dense as water, and boils at 147° C.

The CAS Registry Number for FtBu is 84808-64-0. The CAS name is 1-(1,1-bis(trifluoromethyl)-2,2,2-trifluoroethyl)-1,2,2,3,3,4,4,5,5,6,6-undecafluorocyclohexane. As the FtBu molecule is not asymmetric and has only a single non-fluorine substituent on the cyclohexane ring, the molecule cannot have isomers and thus exists as a single configuration shown as follows:

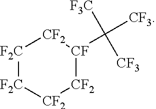

Physical properties of perfluoro(tert-butylcyclohexane) are as follows:

### Molecular Formula C₁₀F₂₀

Molecular Weight (g/mol) 500.08
Physical State @ Room Temp. Liquid
Density (g/mL) 1.97

### Boiling Point (° C.) 147

Vapor Pressure (mmHg) @ 25° C. 3.8
Vapor Pressure (mmHg) @ 37° C. 4.4

### Kinematic Viscosity (cP) 5.378

### Refractive Index @ 20° C. 1.3098

### Calculated Dipole Moment (Debye) 0.287

Calculated Surface Tension (dyne/cm) 14.4

Perfluoro(tert-butylcyclohexane) can carry about 43 mL of oxygen per 100 mL of PFC, and 196 mL of CO₂ per 100 mL of PFC at body temperature.

At room temperature, FtBu is a colorless and odorless liquid that is hydrophobic (virtually insoluble in water) and lipophobic, with only minimal solubility in solvents such as 2,2,4-trimethylpentane (isooctane). FtBu is most soluble in halogenated solvents such as isoflurane. Therefore, FtBu needs to be formulated as an aqueous emulsion for intravenous administration.

FtBu can dissolve and release large amounts of gases, including the blood gases oxygen and carbon dioxide. However, FtBu does not exhibit the oxygen binding properties of hemoglobin, but merely acts as a simple gas solvent. As such, no sinusoidal release curve of oxygen is encountered. The transport and release of oxygen and other gases by FtBu is a simple passive process, the quantity of gas dissolved is linearly related to its partial pressure, essentially following Henry's Law.

### The Perfluoro(Tert-Butylcyclohexane) Emulsion

In one embodiment of the present invention, the PFC selected based on the criteria discussed supra, i.e., perfluoro(tert-butylcyclohexane), is emulsified with a purified surfactant in a buffered, isotonic aqueous medium. The emulsion can contain the list of ingredients as shown in Table 1.

As formulated and manufactured, Oxycyte® is a sterile, non-pyrogenic emulsion consisting of submicron particles (median diameter 200-300 nanometers) of perfluoro(tert-butylcyclohexane) in an aqueous medium that is isotonic and mildly buffered to a neutral pH range. To be physiologically compatible the PFC in Oxycyte® is emulsified with egg-yolk phospholipids. Representative compositions of the PFC emulsion are shown in Tables 1-6.

TABLE 1
Representative PFC Emulsion 1 (60% w/v)

| Component | Function | Mg/mL | % w/v |
|---|---|---|---|
| perfluoro(tert- | Oxygen Carrier | 600.00 | 60.000 |
| butylcyclohexane) | | | |
| Sodium Phosphate | Buffering Agent | 0.57 | 0.057 |
| monobasic Monohydrate | | | |
| Sodium Phosphate Dibasic | Buffering Agent | 3.91 | 0.391 |
| Heptahydrate | | | |
| Glycerin (or NaCl to | Tonicity Adjuster | 13.97 | 1.397 |
| achieve same toxicity) | | | |
| Calcium Disodium Edetate | Trace Metal Scavenger | 0.18 | 0.018 |
| Dihydrate | | | |
| Egg Yolk Phospholipid | Emulsifier/Surfactant | 36.00 | 3.600 |
| Vitamin E (dl-alpha- | Antioxidant | 0.05 | 0.005 |
| tocopherol) | | | |
| Water for Injection | Continuous Phase | 574.83 | 57.483 |
| (WFI) | | | (nominal) |

TABLE 2
Representative PFC Emulsion 2 (60% w/v)

| Component | Function | Mg/mL | % w/v |
|---|---|---|---|
| perfluoro(tert- | Oxygen Carrier | 600.00 | 60.000 |
| butylcyclohexane) | | | |
| Sodium Phosphate | Buffering Agent | 0.47 | 0.047 |
| monobasic Monohydrate | | | |
| Sodium Phosphate Dibasic | Buffering Agent | 3.20 | 0.320 |
| Heptahydrate | | | |
| Glycerin (or NaCl to | Tonicity Adjuster | 11.43 | 1.143 |
| achieve same toxicity) | | | |
| Calcium Disodium Edetate | Trace Metal Scavenger | 0.22 | 0.022 |
| Dihydrate | | | |
| Egg Yolk Phospholipid | Emulsifier/Surfactant | 44.00 | 4.400 |
| Vitamin E (dl-alpha- | Antioxidant | 0.06 | 0.006 |
| tocopherol) | | | |
| Water for Injection | Continuous Phase | 702.57 | 70.257 |
| (WFI) | | | (nominal) |

TABLE 3
Representative PFC Emulsion 3 (60% w/v)

| Component | Function | Mg/mL | % w/v |
|---|---|---|---|
| perfluoro(tert- | Oxygen Carrier | 600.00 | 60.000 |
| butylcyclohexane) | | | |
| Sodium Phosphate | Buffering Agent | 0.06 | 0.006 |
| monobasic Monohydrate | | | |
| Sodium Phosphate Dibasic | Buffering Agent | 0.43 | 0.043 |
| Heptahydrate | | | |
| Glycerin (or NaCl to | Tonicity Adjuster | 1.54 | 0.154 |
| achieve same toxicity) | | | |
| Calcium Disodium Edetate | Trace Metal Scavenger | 0.02 | 0.002 |
| Dihydrate | | | |
| Egg Yolk Phospholipid | Emulsifier/Surfactant | 32.40 | 3.240 |
| Vitamin E (dl-alpha- | Antioxidant | 0.04 | 0.004 |
| tocopherol) | | | |
| Water for Injection | Continuous Phase | 517.35 | 51.735 |
| (WFI) | | | (nominal) |

TABLE 4
Representative PFC Emulsion 4 (60% w/v)

| Component | Function | Mg/mL | % w/v |
|---|---|---|---|
| perfluoro(tert- | Oxygen Carrier | 600.00 | 60.0 |
| butylcyclohexane) | | | |
| Sodium Phosphate | Buffering Agent | 0.52 | 0.052 |
| monobasic Monohydrate | | | |
| Sodium Phosphate Dibasic | Buffering Agent | 3.55 | 0.355 |
| Heptahydrate | | | |
| Glycerin (or NaCl to | Tonicity Adjuster | 12.7 | 1.27 |
| achieve same toxicity) | | | |
| Calcium Disodium Edetate | Trace Metal Scavenger | 0.2 | 0.02 |
| Dihydrate | | | |
| Egg Yolk Phospholipid | Emulsifier/Surfactant | 28.0 | 2.80 |
| Vitamin E (dl-alpha- | Antioxidant | 0.05 | 0.005 |
| tocopherol) | | | |
| Water for Injection | Continuous Phase | 650.7 | 65.07 |
| (WFI) | | | (nominal) |

TABLE 5
Representative PFC Emulsion 5 (60% w/v)

| Component | Function | Mg/mL | % w/v |
|---|---|---|---|
| perfluoro(tert- | Oxygen Carrier | 600.00 | 60.0 |
| butylcyclohexane) | | | |
| Sodium Phosphate | Buffering Agent | 0.52 | 0.052 |
| monobasic monohydrate | | | |
| Sodium Phosphate Dibasic | Buffering Agent | 3.55 | 0.355 |
| Heptahydrate | | | |
| Glycerin (or NaCl to | Tonicity Adjuster | 12.7 | 1.27 |
| achieve same toxicity) | | | |
| Calcium Disodium Edetate | Trace Metal Scavenger | 0.2 | 0.02 |
| Dihydrate | | | |
| Egg Yolk Phospholipid | Emulsifier/Surfactant | 40.0 | 4.0 |
| Vitamin E (dl-alpha- | Antioxidant | 0.05 | 0.005 |
| tocopherol) | | | |
| Water for Injection | Continuous Phase | 638.7 | 63.87 |
| (WFI) | | | (nominal) |

TABLE 6
Representative PFC Emulsion 6 (60% w/v)

| Component | Function | Mg/mL | % w/v |
|---|---|---|---|
| perfluoro(tert- | Oxygen Carrier | 600.00 | 60.000 |
| butylcyclohexane) | | | |
| Sodium Phosphate | Buffering Agent | 0.55 | 0.055 |
| monobasic Monohydrate | | | |
| Sodium Phosphate Dibasic | Buffering Agent | 3.37 | 0.337 |
| Heptahydrate | | | |
| Glycerin (or NaCl to | Tonicity Adjuster | 13.34 | 1.334 |
| achieve same toxicity) | | | |
| Calcium Disodium Edetate | Trace Metal Scavenger | 0.19 | 0.019 |
| Dihydrate | | | |
| Egg Yolk Phospholipid | Emulsifier/Surfactant | 42.00 | 4.200 |
| Vitamin E (dl-alpha- | Antioxidant | 0.05 | 0.005 |
| tocopherol) | | | |
| Water for Injection | Continuous Phase | 670.64 | 67.064 |
| (WFI) | | | (nominal) |

A preferred surfactant used to produce high quality emulsion is a phospholipid mixture that is derived from the yolks of chicken eggs. During the extraction and purification steps of the manufacturing process, the egg phospholipids are rendered non-pyrogenic. Egg phospholipids have a long history of safe use as a surfactant in intravenous lipid emulsions where patient safety is critical.

Egg phospholipid was chosen with this particular phospholipid composition to ensure sufficient stabilization of the interface which forms during the emulsification process. (pure phosphatidyl choline (PC) alone may not be able to sufficiently stabilize this interface) Small percentages of other lipids, particularly lysophosphatidyl choline (LPC) and sphingomyelin (SPH) are present to minimize droplet coalescence and maintain emulsion stability. This influence of emulsifier composition on emulsion stability was previously demonstrated with oil emulsions in general and parenteral fat emulsions specifically. In this formulation, lower concentrations of egg phospholipid may be used down to about 2.5% with the concomitant adjustment of the water amount in the formulation.

The sodium phosphate monobasic monohydrate and sodium phosphate dibasic heptahydrate are chemicals that are used to control the pH of the emulsion formulation. These two chemicals were chosen because phosphate buffers are the most physiologically compatible of the parenteral buffers available. In addition, the minimal buffering capacity the phosphates provide at the formulation amounts is sufficient to maintain a stable emulsion pH range without affecting the natural buffering capacity of the blood. It is important to keep the emulsion pH in a defined range in order to minimize hydrolysis of the egg yolk phospholipids, stabilize the emulsion, and provide a physiologically compatible product.

The pH of this mildly buffered formulation is in the range of 6.8-7.4. This pH range was selected because it represents a good compromise for the phospholipid stability during the shelf life of the emulsion and the median blood pH of 7.2-7.4.

Glycerin USP is used in the formulation to adjust the tonicity of the emulsion. For intravenous infusion, it is important that the tonicity of the emulsion be in the same physiological range as blood tonicity. Glycerin was chosen because it has a long history of use in parenteral emulsions and because it is not an ionizable species that could contribute to coalescence of the emulsion particles by disruption of the charged layer (zeta potential) surrounding the particles. The inventors have conducted experiments which showed that glycerin and mannitol are superior to sodium chloride in terms of mechanical stability of the emulsion.

Calcium disodium edentate dehydrate USP (or disodium edentate USP) is added to the formulation to scavenge any trace metal ions that would accelerate the oxidative degradation of the egg yolk phospholipid surfactant, thereby destabilizing the emulsion.

Vitamin E (dl-alpha-tocopherol) USP is used to dissolve the buffers, tonicity agent and chelating agent to form the continuous phase of the emulsion. Vitamin E belongs to the tocopherol family of natural and synthetic compounds. α-Tocopherol is the most abundant form of this class of compounds. Other members of this class include α-, β-, γ- and δ-tocotrienols. Tocopherols also include α-tocopherol derivatives, such as tocopherol acetate, phosphate, succinate, nicotinate, and linoleate.

In the body the PFC emulsion is capable of uploading and unloading oxygen and CO₂ more efficiently than blood, (at a FtBu concentration of 60% w/v, Oxycyte® can dissolve 3-4 times the amount of oxygen than human hemoglobin can off-load under normal physiological conditions) and this process is concentration-gradient mediated (Henry's Law). Because the median size of the PFC droplets is approximately 40-50 times smaller than an erythrocyte, Oxycyte® is able to oxygenate tissues with narrowed capillaries, as occurs in brain contusions. After about 10 hours, half of an intravenous dose of 3 mL/kg remains in the circulation. PFCs are eliminated from the blood when macrophages scavenge the lipid particles. This is quite similar to how Intralipid® is transported from the blood stream. PFCs are deposited in the liver and spleen. The lipid emulsion is slowly broken down, slowly liberating PFC to be carried back to the lungs on various proteins and lipids wherein the PFC is breathed out as a colorless, odorless and tasteless vapor. In non-human primates, the half-life of PFC in the liver and spleen was found to be dose related; at a dose of 1.8 g/kg (3 mL/kg), the half-life is approximately 12 days.

The PFC emulsions disclosed herein can be used as a vehicle to deliver oxygen to various tissues. To further increase oxygen concentration, the PFC composition can be pre-loaded with molecular oxygen.

It is known that cells need oxygen to regenerate and thrive. Therefore, the PFC emulsion described herein has numerous applications and can be used where oxygen delivery to the cells in a tissue is desired.

### Sickle Cell

As discussed, the PFC emulsion described herein has numerous applications. For example, the PFC emulsion can be used in the treatment of sickle cell disease.

Sickle cell disease (SCD) is a set of genetic abnormalities primarily affecting patients of African and Mediterranean descent. It is caused by a substitution of valine for glutamic acid in the sixth position of the beta globin chain (Agarwal, 2002; Fixler, 2002; Ingram, 1956; Serjeant, 1997). Variations in the disease include homozygous sickle cell anemia (HbSS), compound heterozygous combinations of HbS and thalassemia (HbS-tha1), and heterozygous (HbS-HbC) disease (HbSC). The polymer can alter both the red cell shape and membrane properties leading to abnormal and complex interactions of red cells with the vascular endothelium (Evans, 1987; Noguchi, 1993). The combination of these effects produces a hemolytic anemia and suspected microvascular dysfunction with reductions in microvascular blood flow, the result of which is severe ischemic pain. These episodes of pain have been given the term vasooclusive crisis (VOC). Repetitive episodes of VOC result in acute and chronic end-organ damage which are also pathologically consistent with ischemia and ischemia-reperfusion injury (Bookchin, 1996; Garrison, 1998).

This combination of anemia, reductions in microvascular blood flow, and microvascular dysfunction would appear to make SCD possibly amendable to treatments such as transfusions, modification of rheology, microvascular manipulation using vasodilation, etc. Despite these assumptions, there have been no reported characterizations of oxygen transport in patients with SCD both at baseline and during VOC.

It is shown in Example 4 that sickle cell disease is often accompanied by poor oxygen delivery on a microcirculatory level. Therefore, the PFC emulsion disclosed herein which enhances oxygen delivery to tissues represents a method to ameliorate the symptoms associated with SCD, thereby treating SCD.

### Decompression Sickness

Decompression sickness (DCS) describes a condition arising from the precipitation of dissolved gasses into bubbles inside the body on depressurization. (Vann, 1989) DCS most commonly refers to a specific type of diving hazard but may be experienced in other depressurization events. DCS effects may vary from joint pain and rash to paralysis and death. Treatment is by hyperbaric oxygen therapy (where a patient is entirely enclosed in a pressure chamber breathing 100% oxygen at more than 1.4 times atmospheric pressure) in a recompression chamber. (The Merck Manual, 1999; Leach, 1998; U.S. Navy Diving Manual, 2008) If treated early, there is a significantly higher chance of success.

DCS is caused by a reduction in the ambient pressure surrounding the body, as may happen when leaving a high pressure environment, ascending from depth or ascending to altitude. Depressurization of the body causes excess inert gases, which were dissolved in body liquids and tissues while the body was under higher pressure, to come out of physical solution as the pressure reduces and form gas bubbles within the body. The main inert gas for those who breathe air is nitrogen. The bubbles result in the symptoms of decompression sickness which includes itching skin, rashes, local joint pain and neurological disturbance. The formation of bubbles in the skin or joints results in the milder symptoms, while large numbers of bubbles in the venous blood can cause pulmonary damage. The most severe types of DCS interrupt and damage spinal cord nerve function, leading to paralysis, sensory system failure and death. (The Merck Manual, 1999; Vann, 1989; U.S. Navy Diving Manual, 2008)

Oxygen has traditionally been used to both prevent and treat DCS. One of the most significant breakthroughs in altitude DCS research was oxygen pre-breathing. Breathing pure oxygen before exposure to a low-barometric pressure environment decreases the risk of developing altitude DCS. Oxygen pre-breathing reduces the nitrogen loading in body tissues. Moreover, almost all cases of DCS are initially treated with 100% oxygen until hyperbaric oxygen therapy can be provided. (The Merck Manual, 1999; Leach, 1998; Dehart, 2002; U.S. Navy Diving Manual, 2008)

The PFC emulsion disclosed herein can prevent or treat DCS via a similar mechanism, i.e., quickly transport oxygen into the tissues and reducing nitrogen loading in the body.

### Air Embolism

The PFC emulsion described herein can be used for the treatment of embolism, e.g., surgical iatrogenic air embolism.

An air embolism, or more generally gas embolism, is a physiological condition caused by gas bubbles in a vascular system. In a human body, air embolism refers to gas bubbles in the bloodstream (embolism in a medical context refers to any large moving mass or defect in the blood stream). There are a number of causes for air embolism, e.g., surgical iatrogenesis.

Small amounts of air often get into the blood circulation accidentally during surgery and other medical procedures, e.g., bubbles entering an intravenous fluid line. However, most of these air emboli enter the veins and are stopped at the lungs. Thus, it is rare for a venous air embolism to show symptoms.

However, larger air bubbles in the venous or air embolism in the artery are more serious. For very large venous air embolisms, death may occur if a large bubble of gas becomes lodged in the heart, stopping blood from flowing from the ventricle to the lungs. For arterial gas embolism (AGE), the gas bubble may directly cause stoppage of blood flow to an area bed by the artery, and cause stroke or heart attack if the brain or heart, respectively, are affected.

Hyperbaric oxygen is a traditional first aid treatment for gas embolism. Under hyperbaric conditions, oxygen diffuses into the bubbles, displacing the nitrogen from the bubble and into solution in the blood. Oxygen bubbles are more easily tolerated. Air is composed of 21% oxygen and 78% nitrogen with trace amount of other gases. Additionally, diffusion of oxygen into the blood and tissues under hyperbaric conditions supports areas of the body which are deprived of blood flow when arteries are blocked by gas bubbles. This helps to reduce ischemic injury. Finally, the effects of hyperbaric oxygen antagonize leukocyte-mediated ischemic-reperfusion injury.

Hence, by combining administration of a perfluorocarbon along with oxygen, oxygen can be transported more quickly into the tissues, thereby treating air embolism.

### Carbon Monoxide Poisoning

Carbon monoxide poisoning is the leading cause of death by poisoning in the United States. Each year, approximately 40,000 people seek medical attention for carbon monoxide poisoning, with more than 20,000 visiting the emergency room and more than 4,000 hospitalized. Annually, there are more than 3,800 accidental deaths and suicides caused by carbon monoxide poisoning, with more than 400 Americans dying from unintentional CO poisoning.

Large exposures can lead to significant toxicity of the central nervous system and heart, as well as death. Following acute poisoning, long-term sequelae often occur. However, chronic exposure to low levels of carbon monoxide can also lead to depression, confusion, and memory loss.

Red blood cells (RBCs) pick up carbon monoxide quicker than they pick up oxygen. RBCs have a ˜200 times higher affinity for CO than for O₂. If there is a lot of CO in the air, the body may replace oxygen in the blood with CO, blocking oxygen from getting into the body and causing damage to tissues or death.

Further, CO causes adverse effects in humans by combining with hemoglobin to form carboxyhemoglobin (HbCO) in the blood, poisoning the hemoglobin. This prevents oxygen from binding to hemoglobin, reduces the oxygen-carrying capacity of the blood, and leads to hypoxia. HbCO can revert to hemoglobin but this takes significant time because the HbCO complex is very stable. Symptoms of carbon monoxide poisoning often vary with the percent of HbCo in the blood, and include headache, vertigo, dyspnea, confusion, dilated pupils, convulsions, and coma (The Merck Manual, 1999)

Current treatment of CO poisoning consists of administering 100% oxygen (by breathing mask) or providing hyperbaric oxygen therapy (in pressurized chamber). (The Merck Manual, 1999; Leach, 1998) Oxygen increases the rate of off-loading of carbon monoxide from hemoglobin. In the presence of PFC and the resulting increased concentration of oxygen in the blood, this off-loading of CO may be expedited. By combining administration of a perfluorocarbon along with oxygen, oxygen can be transported more quickly into the oxygen-deprived tissues.

Also, the PFC emulsion would be administered after rescue of a victim who is no longer breathing CO. Since the poisoning of CO is not in the cells but at the hemoglobin level, the PFC would not increase the delivery of CO since once the CO is no longer being inhaled the partial pressure would drop. Therefore, the PFC will not pick up CO and carry it from the lungs. Rather, the PFC would carry O₂ while the hemoglobin is poisoned.

### Traumatic Brain Injury and Spinal Cord Injury

It is known that after Traumatic brain injury (TBI) and spinal cord injury there is an ongoing series of events that leads to tissue damage over time. The initial injury sets up cellular events of calcium flux, ion leakage, cellular apoptosis, vascular insufficiency, neutrophil activation, clot formation, edema etc. All of these mechanisms further feed back into the neuronal apoptosis and cell death mechanisms perpetuating the cycle. The key to intervention, and salvage of individual neurons and axons, is to provide adequate oxygen to the tissues at risk as rapidly as possible after injury. As the cycle of cell death, swelling, apopotosis, edema etc. continues successively more and more cells become injured and die. Thus, the sooner one can intervene with oxygen delivery to cells at risk, the quicker and greater numbers of cells are saved. In the central nervous system (CNS), tissue cells die quickly when all oxygen is removed. Each cell that dies can translate into a circuit unable to be completed. CNS tissue cannot, at the present time, be regenerated by medical intervention. Early intervention to salvage the maximum number of cells represents a way to decrease the severity of injury and improve outcome for the patients.

Approximately ⅓ of severe head injury patients show reduced oxygen tension during the first 6 to 24 hours following injury, often due to reduced cerebral blood flow (CBF) caused by e.g. narrowed vessels, which can lead to post-traumatic brain damage and a significantly worse outcome (Zauner, 1997; Zauner, 1997). Thus, the prevention of secondary ischemia by the enhancement of early O₂ delivery should be of great benefit (Kwon, 2005). The PFC emulsion can dramatically enhances oxygen delivery from red blood cells to tissues. PFC emulsions are also made up of pure PFC inside lipid membranes with a particle size far smaller than erythrocytes. Because of the small particle size, coupled with enhanced oxygen diffusivity, oxygen can be delivered to tissues with very low, trickle, flow. PFC is known to increase cerebral blood flow and also to decrease inflammatory reactions. Also, PFC has enhanced gas carrying capacity for CO₂ as well as nitric oxide. These research observations may play roles in salvaging injured central nervous system cells.

It should be noted that PFC emulsions deliver even more gas when cooled. Therefore, the utilization of cooling of the PFC emulsion prior to or during the act of infusion into the body may also be an adjunct and part of the invention disclosure as well.

### Organ Preservation and Restoration of Organ Function

Due to a shortage of organs, more and more cadaver organs are being used in transplant. The duration of the time the organ is kept on ice and without a blood supply should be kept to a minimum but the time often becomes lengthy and organ survival decreases. By perfusing the organ with the PFC composition described herein, the organ can survive for a longer period of time without a blood supply and is better preserved prior to transplant.

The emulsion could bath the organ as well as be perfused through it during transport/prior to surgery, thereby providing a constant source of oxygen that will help preserve the organ and reduce the incidence of reperfusion injury once the organ is transplanted. The emulsion should also help with graft acceptance for many of the same reasons discussed herein, e.g., promotion of faster cell repair and angiogenesis.

### Topical Indications

Although the PFC emulsions described herein are primarily formulated for intravenous use, they can also be used for topical indications. These topical indications include: wound and burn healing, scar prevention and reduction, enhancement of sexual function, treatment of acne and rosacea, and cosmetic use, including promotion of anti-aging.

### Other Indications and Uses

Other indications and uses for the PFC emulsion described herein include: use as air deodorizer, treatment of canker sores, treatment of cavities, use in chemotherapy and radiation treatment, treatment of constipation, use as imaging contrasting agent, treatment of decubitus ulcer, use in detoxification and colon cleansing, treatment of diabetic foot care, treatment off gas gangrene, treatment of hemorrhoids, use in fighting intestine infection caused by Clostridium difficile, treatment for intestinal parasites for humans and animals, treatment of muscle pain/aching muscle, treatment of nocturnal leg cramps, use for pruritus relief and providing faster healing of irritated skin, use in shampoo, conditioner, dandruff or hair loss products to provide oxygen to hair, and use to accelerate skin graft uptake/increase skin graft survival.

The perfluorocarbon employed in the compositions and methods described herein may be in compositions which may further comprise pharmaceutically acceptable carrier or cosmetic carrier and adjuvant(s) suitable for intravenous, intra-arterial, intravascular, intrathecal, intratracheal or topical administration. Compositions suitable for these modes of administration are well known in the pharmaceutical and cosmetic arts. These compositions can be adapted to comprise the perfluorocarbon or oxygenated perfluorocarbon. The composition employed in the methods described herein may also comprise a pharmaceutically acceptable additive.

The perfluorocarbon emulsions disclosed herein can comprise excipients such as solubility-altering agents (e.g., ethanol, propylene glycol and sucrose) and polymers (e.g., polycaprylactones and PLGA's) as well as pharmaceutically active compounds.

The perfluorocarbon emulsions of the methods, uses and pharmaceutical compositions of the invention may include perfluorocarbon-in-water emulsions comprising a continuous aqueous phase and a discontinuous perfluorocarbon phase. The emulsions typically include emulsifiers, buffers, osmotic agents, and electrolytes. The perfluorocarbons are present in the emulsion from about 5% to 130% w/v. Embodiments include at least about 40%, 45%, 50%, 55%, 60%, 65%, 70%, 75%, 80% and 85% w/v. A 60% w/v F-tert-butylcyclohexane emulsion may be used as the perfluorocarbon emulsion in one embodiment. Embodiments also include an egg yolk phospholipid emulsion buffered in an isotonic medium wherein the perfluorocarbon is present in the emulsion from about 5% to 130% w/v.

The multiplicity of configurations may contain additional beneficial biologically active agents which further promote tissue health.

The compositions of this invention may be administered in forms detailed herein. The use of perfluorocarbon may be a component of a combination therapy or an adjunct therapy. The combination therapy can be sequential or simultaneous. The compounds can be administered independently by the same route or by two or more different routes of administration depending on the dosage forms employed. The dosage of the compounds administered in treatment will vary depending upon factors such as the pharmacodynamic characteristics of a specific therapeutic agent and its mode and route of administration; the age, sex, metabolic rate, absorptive efficiency, health and weight of the recipient; the nature and extent of the symptoms; the kind of concurrent treatment being administered; the frequency of treatment with; and the desired therapeutic effect.

A dosage unit of the compounds may comprise a single compound or mixtures thereof with other compounds. The compounds can be introduced directly into the targeted tissue, using dosage forms well known to those of ordinary skill in the cosmetic and pharmaceutical arts.

The compounds can be administered in admixture with suitable pharmaceutical diluents, extenders, excipients, or carriers (collectively referred to herein as a pharmaceutically acceptable carrier) suitably selected with respect to the intended form of administration and as consistent with conventional pharmaceutical and cosmetic practices. The compounds can be administered alone but are generally mixed with a pharmaceutically acceptable carrier. This carrier can be a solid or liquid, and the type of carrier is generally chosen based on the type of administration being used. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents.

Techniques and compositions for making dosage forms useful in the present invention are described in the following references: Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol. 40 (Gilbert S. Banker, Christopher T. Rhodes, Eds.). All of the

The PFC compositions may contain antibacterial agents which are non-injurious in use, for example, thimerosal, benzalkonium chloride, methyl and propyl paraben, benzyldodecinium bromide, benzyl alcohol, or phenylethanol.

The PFC compositions may also contain buffering ingredients such as sodium acetate, gluconate buffers, phosphates, bicarbonate, citrate, borate, ACES, BES, BICINE, BIS-Tris, BIS-Tris Propane, HEPES, HEPPS, imidazole, MES, MOPS, PIPES, TAPS, TES, and Tricine.

The PFC compositions may also contain a non-toxic pharmaceutical organic carrier, or with a non-toxic pharmaceutical inorganic carrier. Typical of pharmaceutically acceptable carriers are, for example, water, mixtures of water and water-miscible solvents such as lower alkanols or aralkanols, vegetable oils, peanut oil, polyalkylene glycols, petroleum based jelly, ethyl cellulose, ethyl oleate, carboxymethyl-cellulose, polyvinylpyrrolidone, isopropyl myristate and other conventionally employed acceptable carriers.

The PFC compositions may also contain non-toxic emulsifying, preserving, wetting agents, bodying agents, as for example, polyethylene glycols 200, 300, 400 and 600, carbowaxes 1,000, 1,500, 4,000, 6,000 and 10,000, antibacterial components such as quaternary ammonium compounds, phenylmercuric salts known to have cold sterilizing properties and which are non-injurious in use, thimerosal, methyl and propyl paraben, benzyl alcohol, phenyl ethanol, buffering ingredients such as sodium borate, sodium acetates, gluconate buffers, and other conventional ingredients such as sorbitan monolaurate, triethanolamine, oleate, polyoxyethylene sorbitan monopalmitylate, dioctyl sodium sulfosuccinate, monothioglycerol, thiosorbitol, ethylenediamine tetracetic.

The PFC compositions may also contain surfactants that might be employed include polysorbate surfactants, polyoxyethylene surfactants, phosphonates, saponins and polyethoxylated castor oils, but preferably the polyethoxylated castor oils. These surfactants are commercially available. The polyethoxylated castor oils are sold, for example, by BASF under the trademark Cremaphor.

The PFC compositions may also contain wetting agents commonly used in ophthalmic solutions such as carboxymethylcellulose, hydroxypropyl methylcellulose, glycerin, mannitol, polyvinyl alcohol or hydroxyethylcellulose and the diluting agent may be water, distilled water, sterile water, or artificial tears, wherein the wetting agent is present in an amount of about 0.001% to about 10%.

The formulation of this invention may be varied to include acids and bases to adjust the pH; tonicity imparting agents such as sorbitol, glycerin and dextrose; other viscosity imparting agents such as sodium carboxymethylcellulose, microcrystalline cellulose, polyvinylpyrrolidone, polyvinyl alcohol and other gums; suitable absorption enhancers, such as surfactants, bile acids; stabilizing agents such as antioxidants, like bisulfites and ascorbates; metal chelating agents, such as sodium edetate; and drug solubility enhancers, such as polyethylene glycols. These additional ingredients help make commercial solutions with adequate stability so that they need not be compounded on demand.

Other materials as well as processing techniques and the like are set forth in Part 8 of Remington's Pharmaceutical Sciences, 17th edition, 1985, Mack Publishing Company, Easton, Pa., and International Programme on Chemical Safety (IPCS), which is incorporated herein by reference.

It is understood that where a parameter range is provided, all integers within that range, and tenths thereof, are also provided by the invention. For example, “20-80% w/v” includes 20.0% w/v, 20.1% w/v, 20.2% w/v, 20.3% w/v, 20.4% w/v etc up to 80.0% w/v.

All combinations and sub-combinations of the various elements of the methods described herein are envisaged and are within the scope of the invention.

This invention will be better understood by reference to the Experimental Details which follow, but those skilled in the art will readily appreciate that the specific experiments detailed are only illustrative of the invention as described more fully in the claims which follow thereafter.

### EXPERIMENTAL DETAILS

### Example 1

### Manufacturing the PFC Emulsion

It is vital that emulsion particles intended for intravenous administration are small and uniform in order to enable the particles to pass through the microcirculation. The inventors have found that the process steps used to manufacture the emulsion are critical to achieve a size distribution of particles that are small, stable, and physiologically compatible. As such the particle size and particle size distribution are important characteristics of the emulsion. To obtain these characteristics in a reproducible manner, both emulsification steps, coarse and, high pressure, should be controlled. These emulsion characteristics depend strongly on the energetics of the coarse emulsification process which, in turn, depends greatly on the size and speed of the emulsification tool as well as on the rate of the PFC addition to the aqueous dispersion.

The inventors have found that an ideal coarse emulsion is monomodal with a median particle size of less than 20 micrometers.

The inventors have also found that such a coarse emulsion with ideal characteristics is preferred because upon further processing with high pressure homogenization, it is most likely that a stable “final” emulsion is produced. Such an emulsion is characterized by a narrow monomodal distribution centered around 200-300 nanometers without a substantial population of undesirable larger size (>10 micrometers) particles.

Specialized equipment is used in the manufacturing of the PFC emulsion. The manufacturing process steps should be performed in a specific sequence to produce an emulsion with desirable/optimal characteristics.

A pilot-scale 8 liter batch of the PFC emulsion disclosed herein is manufactured according to the methods set out below:

### Manufacturing Equipment

PFC Addition Vessel: A PFC addition vessel is used to deoxygenate the perfluorocarbon and to transfer the perfluorocarbon to a processing vessel containing the remainder of the emulsion formulation ingredients.

Mixing Vessel: A mixing vessel is a container into which all of the formulation ingredients are added together, dissolved or dispersed, and mixed under high shear to create a coarse emulsion. The preferred vessel is a water-jacketed stainless steel cylindrical vessel whose temperature is controlled by circulating water from a thermostatted water bath through the vessel jacket. The mixing vessel contains a central port in the top to accommodate a high shear mixing shaft and blade.

High Shear Mixer: A high shear mixer equipped with a rotor/stator dispersing element is preferred for high shear mixing of the formulation ingredients to create a coarse emulsion with all of the formulation ingredients in the mixing vessel prior to the high pressure homogenization process.

Homogenization Vessels: For the homogenization step in the manufacture of emulsion, two processing vessels equipped with mechanical stirrers are used in either of two configurations. In the first configuration one vessel is used as a circulation vessel. The other vessel serves as a filling vessel. In the second configuration both processing vessels are used in a discrete pass setup in which the vessels alternate feeding emulsion to the inlet of the homogenizer and receive material from the outlet of the homogenizer.

Homogenizer: Preferably a suitably equipped 2-stage homogenizer is used for the homogenization step of the emulsion manufacturing process.

Transfer Lines & Tubing: Stainless steel, high density polyethylene, or polypropylene tubing should be used for all transfer lines that come into contact with the emulsion. Silicone tubing is not acceptable for use in the manufacturing process due to potential incompatibilities with the perfluorocarbon.

In-line Process Filter: A 10-μm cartridge filter is used for filtration of particulate matter from the emulsion just prior to filling. These filters should be compatible with the emulsion and minimize shear forces that may remove a portion of the surfactant coating from the emulsion particles.

Sterilizer (Autoclave): It is an FDA requirement that all emulsions intended for intravenous administration be sterile. Because of the relatively broad droplet size distributions found in perfluorocarbon emulsions, and the potential fragility of the droplets when forced through a fine filter under pressure, sterile filtration techniques using 0.22 micron filters is not used. Therefore, the emulsion is subjected to terminal heat sterilization in a steam autoclave. A rotary-drum steam autoclave is preferred to ensure even heat distribution of the emulsion product as it is terminally sterilized because of the large difference in heat capacity between the perfluorocarbon and the water in the emulsion formulation.

### Example 1A

### FtBu Emulsion

### Manufacturing Process Steps

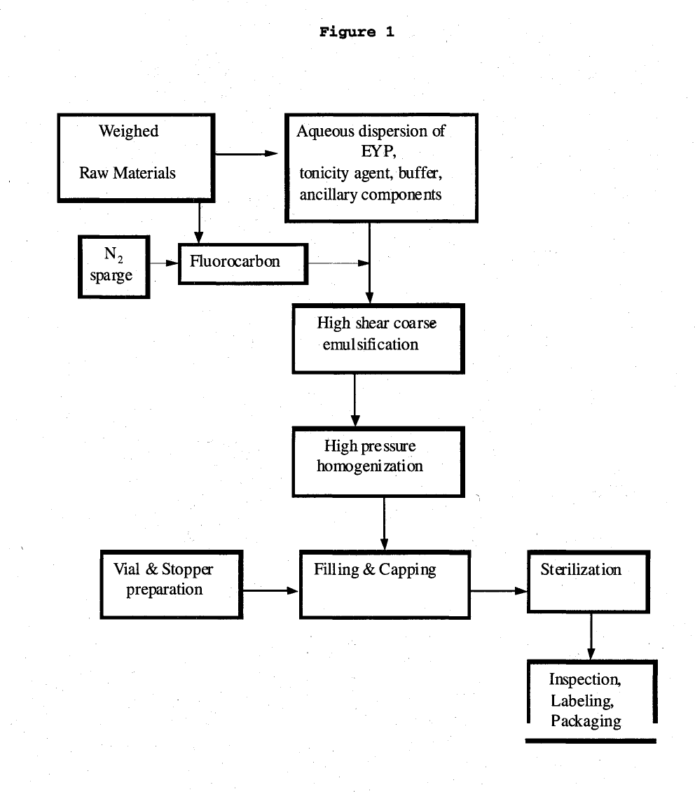

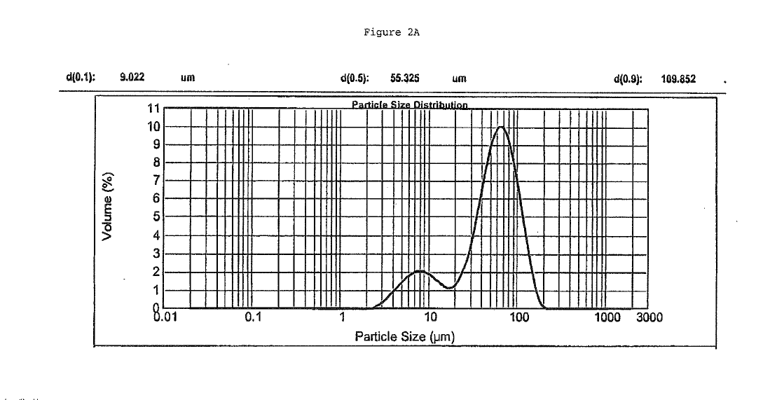

The PFC Emulsion (60% w/v) described herein is manufactured according to the process shown in FIG. 1.

An inert blanketing gas such as nitrogen is used to blanket the emulsion during the manufacturing process and blanket the headspace of the product vials prior to capping in order to minimize phospholipid degradation during shelf storage.

### Perfluorocarbon Deoxygenation

In a separate step that precedes the compounding of formulation ingredients, the weighed perfluorocarbon is placed into the PFC addition vessel in which it is continuously sparged with nitrogen gas through a fritted glass or stainless steel tube extending into the bottom of the perfluorocarbon to remove dissolved oxygen.

### Addition and Dispersion of Ingredients

Under a nitrogen blanket, the required amount of Water for Injection (WFI) is added to the water-jacketed stainless steel mixing vessel that is fitted with a high shear mixer and rotor/stator dispersing element. The WFI is then heated to 50-55° C. before any of the remaining formulation ingredients are added. When the temperature of the WFI reaches the desired temperature, the high shear mixer is turned on and set at low speed. The formulation ingredients are then added to the WFI in the mixing vessel in the following order: NaH₂PO₄.H₂O, Na₂HPO₄.7H₂O, CaNa₂EDTA.2H₂O, and glycerin.

Nitrogen blanketing of the headspace and mixing are continued throughout the addition and dispersion of the remaining formulation ingredients.

At this point in the process, the egg yolk phospholipid is removed from the freezer and then quickly weighed into a transfer container that was previously cooled to −20° C. or lower and quickly added to the mixing vessel. These precautionary steps are taken to minimize exposure of the egg phospholipid to heat and oxygen and to enable efficient transfer of the phospholipid before it absorbs moisture and becomes sticky.

The Vitamin E is now weighed and added to the mixing vessel.

After the addition of the egg yolk phospholipid and Vitamin E, the high shear mixer speed is increased to mid-range and mixing is continued until the phospholipid is adequately dispersed.

### Perfluorocarbon Addition & High Shear Coarse Emulsification

The high shear mixer is set at maximum speed and the vessel contents are thermostatted at 50-55° C. The perfluorocarbon is added at a rate of approximately 50-100 mL/minute (or less) from the PFC addition vessel to the mixing vessel through a stainless steel transfer line that terminates near the rotor-stator blades of the mixer. Mixing is continued under a nitrogen blanket to thoroughly disperse the perfluorocarbon and form a coarse emulsion. During this mixing period a sample of the coarse emulsion is withdrawn for a particle size distribution (PSD) measurement.

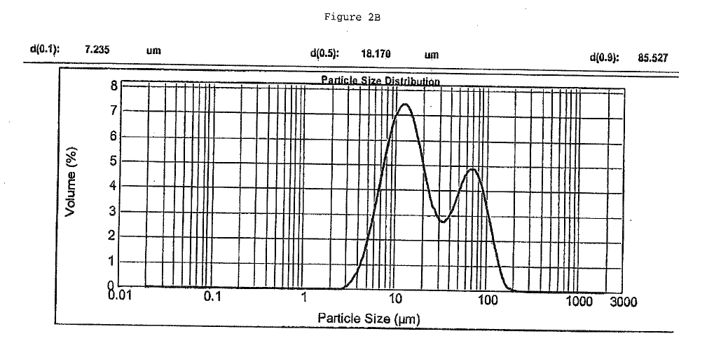

At this point for the PSD of the coarse emulsion should be monomodal with a median particle size less than 20 micrometers. The criteria for the PSD of the coarse emulsion are important because the inventors have found that the presence of a second population of larger particles will persist even after high pressure homogenization, resulting in a failure to meet particle size specifications based on physiological requirements. Various coarse emulsion PSDs are shown in FIGS. 2-5.

FIG. 2A shows an unacceptable coarse emulsion after PFC addition. FIG. 2A shows bimodal distribution with modes at 8.2 and 65 micrometers.

FIG. 2B shows the same coarse emulsion after having been subjected to additional high shear mixing. The amount of undesirable larger size particles has been reduced but not eliminated.

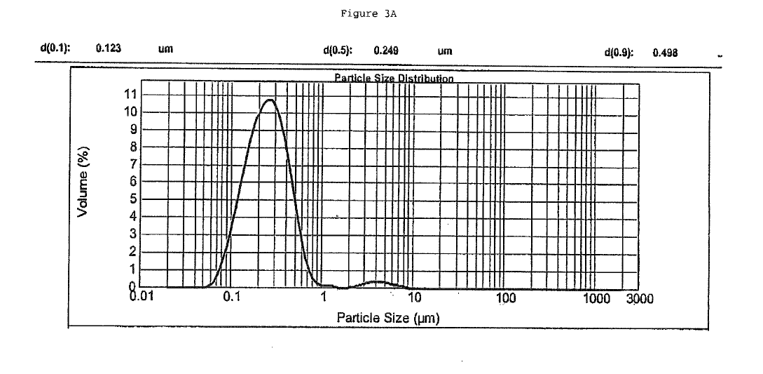

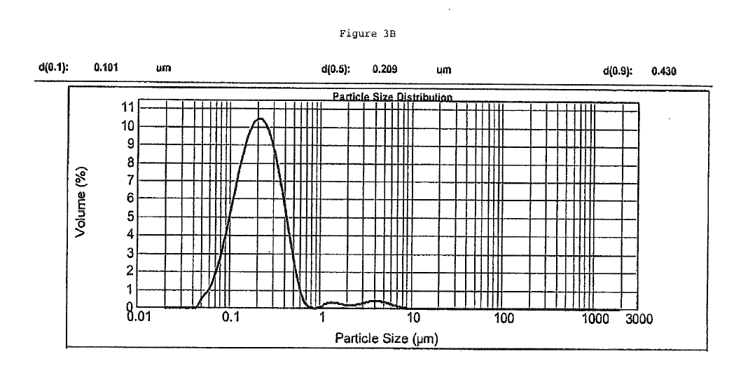

FIG. 3A shows the PSD of the coarse emulsion seen in FIG. 2B after the emulsion has been subjected to high pressure homogenization. A second population centered near 4 micrometers is still present.

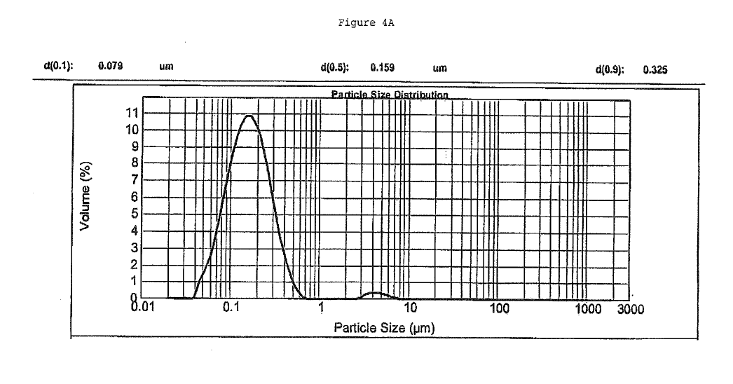

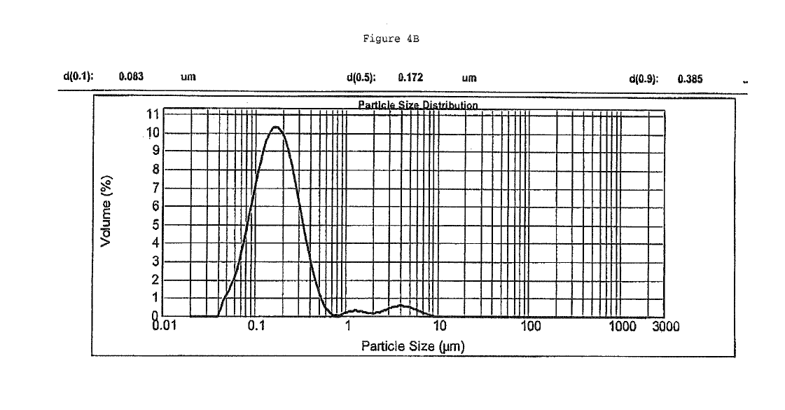

Additional homogenization time does not eliminate this second population, nor does increasing homogenization pressure, as can be seen in FIGS. 3-4.

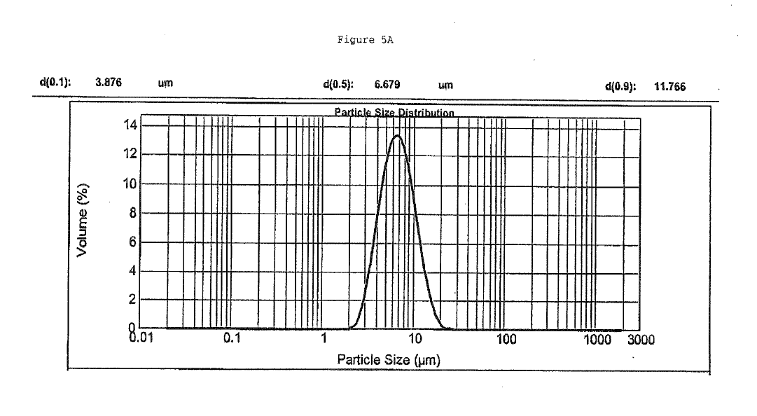

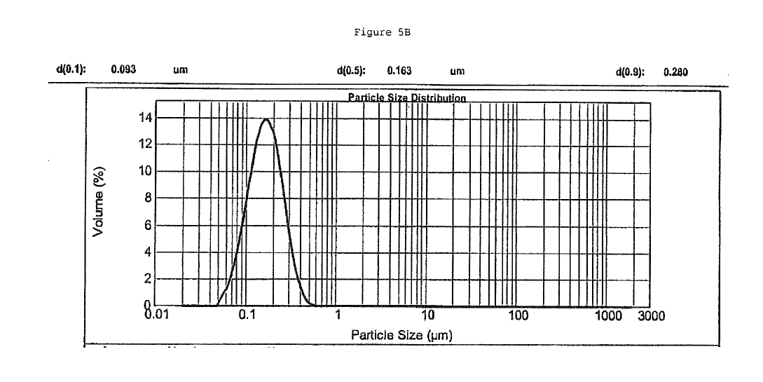

FIG. 5A shows the PSD of an acceptable coarse emulsion prior to high pressure homogenization. This distribution is monomodal with a mode centered at 6 micrometers. High pressure homogenization of this coarse emulsion resulted in the monomodal, small particle size distribution shown in FIG. 5B.

After the high shear mixing is complete, in-process testing of the particle size distribution and the pH of the coarse emulsion is performed before proceeding to the high pressure homogenization. Droplet size is measured to assure that the succeeding homogenization step produces small emulsion droplets and as narrow a distribution as possible with batch-to-batch consistency. The pH should be in the range of 6.8-7.4 because as emulsion droplets decrease in size, they adsorb hydroxide ions into a near-film layer which is a stabilizing influence. Values of pH outside this range can be detrimental to phospholipid and ultimately emulsion stability.

### Homogenization

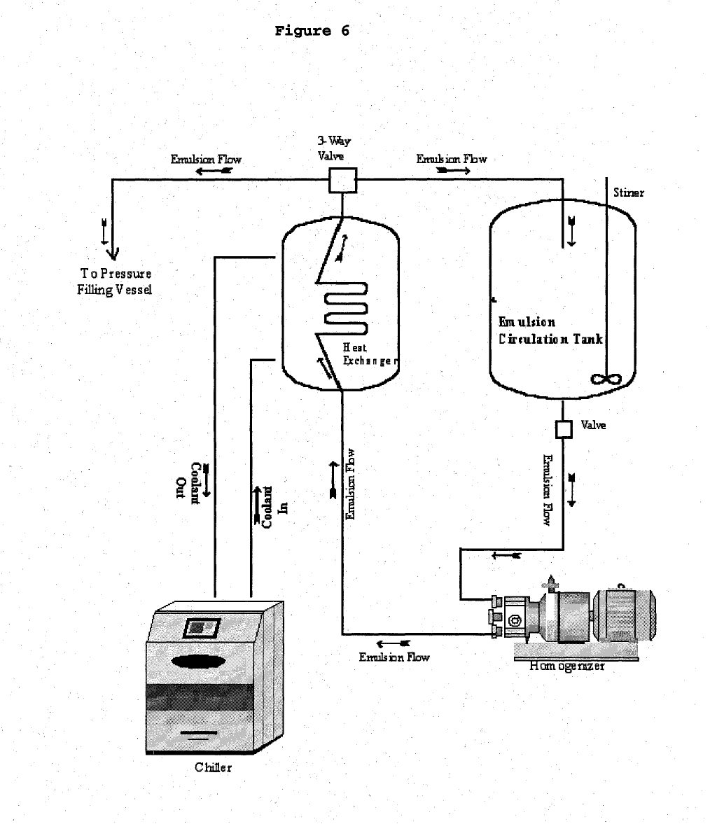

The coarse emulsion is transferred, preferably through a stainless steel line under nitrogen pressure, from the mixing vessel to a stainless steel receiving vessel. This receiving vessel is a component of either a recirculation homogenization set-up (sample set up shown in FIG. 6) or a discrete pass homogenization set-up. Both set-ups use a heat exchanger between the outlet of the homogenizer and the inlet of the receiving vessel.

The circulating vessel is equipped with a low speed stirrer and the headspace in the vessel is continuously blanketed with nitrogen. The temperature of the chilling water in the heat exchanger is maintained at 11-15° C. The inventors have found that very low processing temperatures are detrimental to obtaining a small-particle emulsion. The coarse emulsion is continuously circulated through the homogenizer at a pressure of 8,000-9,000 psi (stage 2 valve set to 800-900 psi) for a time equivalent to at least 3-6 discrete passes. The emulsion in the circulation vessel is stirred at low speed during the entire homogenization process to avoid sedimentation.

The emulsification time is dependent on batch size and flow rate through the homogenizer and is determined from a continuous flow calculation (Leviton, 1959). A homogenization process using a discrete pass set-up usually requires less processing than a continuous pass approach.

In the continuous recirculation set-up, after the calculated amount of time, the product flow is directed to the stainless steel filling vessel, and the homogenizer is used as a pump to transfer the emulsion over to this vessel for filling. During the transfer process, the emulsion is continuously stirred at low speed and the vessel atmospheres are continuously blanketed with nitrogen.

### Filling and Capping

The filling vessel is pressurized with nitrogen and the emulsion passes from the filling vessel with nitrogen pressure through a 10-μm in-line filter (to remove particulates) to a filling nozzle and into depyrogenated glass bottles. The filter should be compatible with the emulsion and minimize shear forces that could strip a portion of the surfactant coating from the emulsion droplets.

The optimum fill volume is chosen such that 1) the stoppers do not push out during autoclaving 2) sufficient headspace prevents “microdistillation” of the perfluorocarbon during autoclaving. The bottle headspace is blanketed with nitrogen, the bottles are stoppered, and sealed with aluminum crimp seals using a qualified capper.

Sterilization

After filling is completed the filled bottles are placed into sterilizer racks and terminally sterilized in a rotary steam autoclave using a customized sterilization cycle that is validated to ensure product sterility while maintaining product integrity.

### PFC Emulsion Stability

The ideal emulsion should continue to meet all of the initial acceptance specifications during its intended shelf life. The particle size and particle size distribution differ from other specifications because they will change as the emulsion ages. This growth is inevitable because the emulsion, by definition, is thermodynamically unstable. Even a good emulsion will exhibit some growth in particle size during its intended shelf life, whether by Ostwald ripening, coalescence, flocculation, or sedimentation. However, if the emulsion is properly formulated and the manufacturing process is optimized, the particle size growth rate should be reasonably small, the median size should remain in the 200-400 nm range, and the particle size distribution should remain reasonably narrow.

FIG. 5 shows a representative particle size distribution of a good PFC emulsion (60% w/v), as measured by a laser light scattering technique (Malvern Mastersizer) liquid-phase photosedimentation technique (Horiba CAPA 700).

These graphical representations of the particle size data provide clear evidence of the submicron nature of the perfluorocarbon emulsions. Further, measurements obtained by laser diffraction and photocorrelation spectroscopy indicate that over 99% of the emulsion particles are less than 1 μm in diameter. Photomicroscopy data generated by the inventor also support the absence of larger-sized

Thus, the FtBu emulsion manufactured in accordance with the above-described procedure is reasonably stable and has the following characteristics:
1. The FtBu emulsion contains less than 20 ppm residual fluoride by weight of the emulsion;
2. The FtBu emulsion contains less than 7 g/L lysophosphatidylcholine (LPTC) by weight of the emulsion;
3. The FtBu emulsion contains less than 1 ppm residual conjugated olefin by weight of the FtBu;
4. The FtBu emulsion contains less than 0.7 ppm residual fluoride by weight of the FtBu;
5. The FtBu emulsion contains less than 5 ppm residual organic hydrogen by weight of the FtBu;
6. The FtBu emulsion has D(0.9) value of about 600 nm; and
7. The FtBu emulsion has D(0.5) value of about 200-330 nm.

### Example 1B

### Perfluorodecalin Emulsion

An emulsion comprising perfluorodecalin is manufactured following the procedure described in Example 1A. The resulting perfluorodecalin emulsion is reasonably stable and has the following characteristics:
1. The Perfluorodecalin emulsion contains less than 20 ppm residual fluoride by weight of the emulsion;
2. The Perfluorodecalin emulsion contains less than 7 g/L lysophosphatidylcholine (LPTC) by weight of the emulsion;
3. The Perfluorodecalin emulsion contains less than 1 ppm residual conjugated olefin by weight of the Perfluorodecalin;
4. The Perfluorodecalin emulsion contains less than 0.7 ppm residual fluoride by weight of the Perfluorodecalin;
5. The Perfluorodecalin emulsion contains less than 5 ppm residual organic hydrogen by weight of the Perfluorodecalin;
6. The Perfluorodecalin emulsion has D(0.9) value of about 600 nm; and
7. The Perfluorodecalin emulsion has D(0.5) value of about 200-330 nm.

### Example 1C

### Perfluorooctylbromide Emulsion

An emulsion comprising perfluorooctylbromide is manufactured following the procedure described in Example 1A. The resulting perfluorooctylbromide emulsion is reasonably stable and has the following characteristics:
1. The Perfluorooctylbromide emulsion contains less than 20 ppm residual fluoride by weight of the emulsion;
2. The Perfluorooctylbromide emulsion contains less than 7 g/L lysophosphatidylcholine (LPTC) by weight of the emulsion;
3. The Perfluorooctylbromide emulsion contains less than 1 ppm residual conjugated olefin by weight of the Perfluorooctylbromide;
4. The Perfluorooctylbromide emulsion contains less than 0.7 ppm residual fluoride by weight of the Perfluorooctylbromide;
5. The Perfluorooctylbromide emulsion contains less than 5 ppm residual organic hydrogen by weight of the Perfluorooctylbromide;
6. The Perfluorooctylbromide emulsion has D(0.9) value of about 600 nm; and
7. The Perfluorooctylbromide emulsion has D(0.5) value of about 200-330 nm.

### Example 1D

### Dodecafluoropentane (DDFP) Emulsion

An emulsion comprising DDFP is manufactured following the procedure described in Example 1A. The resulting DDFP emulsion is reasonably stable and has the following characteristics:
1. The DDFP emulsion contains less than 20 ppm residual fluoride by weight of the emulsion;
2. The DDFP emulsion contains less than 7 g/L lysophosphatidylcholine (LPTC) by weight of the emulsion;
3. The DDFP emulsion contains less than 1 ppm residual conjugated olefin by weight of the DDFP;
4. The DDFP emulsion contains less than 1 ppm residual fluoride by weight of the DDFP;
5. The DDFP emulsion contains less than 10 ppm residual organic hydrogen by weight of the DDFP;
6. The DDFP emulsion has D(0.9) value of about 600 nm; and
7. The DDFP emulsion has D(0.5) value of about 200-300 nm.

### Example 2

Oxycyte® emulsion (60% w/v PFC) was tested systemically via intravenous administration at various dosages to Sprauge Dawley rats, Cynomolgus Monkeys and, humans.

The Oxycyte® emulsion was found to be well tolerated and had no toxicity.

### Example 3

### Measuring Oxygen Tension in Tissue

A material which binds oxygen (fluorescent marker) is injected into skin tissue. The combination is fluorescent and the more oxygen that is present, the stronger the fluorescent signal. (representing the oxygen tension in the tissue).

First it is determined that fluorescence chemistry is unaffected by the PFCs and poloxamers. Then as a control, the fluorescent marker is injected into the skin, and oxygen tension is obtained. Finally, the same area is treated with a PFC, PFC emulsion or a PFC gel and oxygen tension is again obtained.

Result: oxygen tension reading begins to spike after injection of the marker into the area treated with PFC, then starts to decline as the PFC is eliminated from the tissue.

Conclusion: the absorption of an oxygen-binding PFC like FtBu or APF-200 substantially increases local oxygen tension in the tissue. The resulting increase in local oxygen concentration may serve both to increase rates of wound healing and rates of free-radical deactivation.

### Example 4

### Sickle Cell Disease Ischemia

### Example 4A

Better characterization of sickle cell disease (SCD) and vaso-occlusive crisis (VOC) was sought using a number of new noninvasive measurements of both local and global oxygen transport. These include simultaneous measurements of, oxygen delivery (DO₂), and tissue oxygenation and surrogates of oxygen consumption such as the oxygen extraction ratio (OER). These techniques were used along with conventional hemodynamic parameters such as heart rate and blood pressure to measure and compare oxygen transport and hemodynamics in SCD patients at baseline, SCD patients in VOC, and patients with no SCD.

### Study Population

The study population consisted of three groups. The first was twenty normal healthy controls of African-American descent with no prior history of sickle cell disease or trait. These patients also reported no past medical history for chronic disease including hypertension, diabetes, or coronary artery disease and were not taking medicines for any condition. The second group consisted of forty-four SCD patients with a known history of homozygous Hb SS or doubly heterozygous Hb S-βTha1 or Hb SC disease who at the time of evaluation did not report pain. The last group was seventeen sickle cell patients with a verified history of Hb SS or Hb SC disease who at the time of evaluation reported symptoms consistent with a VOC which required treatment in the emergency department. Genotype was verified through chart review.

### Noninvasive Hemodynamic and Oxygen Utilization Measurements

Cutaneous Tissue Hemoglobin Oxygen Saturation Measurements (CtSO₂): Differential absorption spectroscopy was used to measure the aggregate hemoglobin oxygen saturation in a selected volume of tissue. CtSO₂ measurements were made with a spectrophotometric (Wolff, 1998; Woff, 1996) monitor using visible light (500-700 nm) to detect CtSO₂ (O2C: LEA, Inc., Gie [symbol] en, Germany). Oxygen saturation was determined by differential absorption spectra of oxy- and deoxyhemoglobin to the light as it traverses a certain volume of tissue. The volume of blood distributed in any tissue is approximately 80% venous, 10% capillary, and 10% arterial (Guyton, 1981). The derived CtSO₂ is thus indicative of mainly venous hemoglobin and thus the post-extraction compartment of the tissue. This in turn is indicative of the adequacy of oxygen delivery at the tissue level. This is the basis for current near infrared absorption spectroscopy technology for the measurement of peripheral tissue and brain hemoglobin oxygen saturation (Ward, 2006). The combination of the wavelengths of light used, as well as optode spacing, limits the source of the returning signal to a depth of 2 mm. At this depth subcutaneous tissue is being interrogated and not deeper tissues such as muscle. One flat probe was secured to the thenar aspect of the palmar surface of one hand (to minimize any effect of pigment and adipose effects noted in prior evaluations) during the recording of CtSO₂ data. CtSO2 was measured continuously and values (reported as percent saturation) were recorded every 5 seconds for averaging over the 10 minute period. CtSO2 is reported as % hemoglobin oxygen saturation.

Arterial Hemoglobin Oxygen Saturation: Arterial hemoglobin oxygen saturation (SpO₂) was determined with the use of a pulse oximeter (General Electric Procare Auscultaroy 400). SpO₂ was used to substitute for true arterial hemoglobin oxygen saturation. SpO₂ was measured every 5 seconds and averaged over the 10 minute monitoring period.

Tissue Microvascular Oxygen Extraction Ratio (OERM): OERM is an indicator of the degree to which oxygen is being extracted and thus is an indicator of the balance between oxygen delivery and consumption. It can be determined by several methods both globally and regionally. Globally this measure is usually calculated as VO₂/DO₂ or more commonly as mixed venous hemoglobin oxygen saturation divided by arterial hemoglobin oxygen saturation. For this study we localized OERM was determined by utilizing the CtSO₂ as an indicator of tissue venous hemoglobin oxygen saturation and SpO₂ as the indicator of tissue arterial hemoglobin oxygen saturation. In order to account for the distribution of venous blood within the volume of tissue being interrogated the following formula was used: 0.8×CtSO₂/SpO₂, where 0.8 is a factor accounting for the degree of venous distribution of blood volume within the tissue (Guyton, 1981; Ward, 2006; Hogan, 2007).

Cardiac Index (CI): Cardiac Index, which was indexed to body surface area (BSA), was measured using an impedance cardiography (Pennock, 1997; Van De Water, 2003) (Medis Medizinische Me [symbol] technik, Thueringen, Germany). Eight standard electrodes were placed on each subject as directed by the manufacturer. Two of these electrodes are place on each side of the neck and thorax. The electrodes used were standard continuous ECG monitoring electrodes. CI was measured every 5 seconds and these values were used to average CI over the 10 minute period. Variables measured using impedance cardiography included, cardiac output, stroke volume, and stoke index (also indexed to BSA).

Oxygen Delivery: Oxygen delivery was calculated (DO₂I=CI*(13.4*Hgb*O₂SAT)) (Tobin, 1998). Hemoglobin was measured as part of the routine clinic visits or Emergency Department visits. Control subjects did not have hemoglobin levels drawn. A standard hemoglobin value of 12 or 14 was used for the control subjects. Hemoglobin of 12 for women and 14 for men was chosen for calculating oxygen delivery because this number represents the low range of normal hemoglobin levels and would underestimate oxygen delivery in our control patients.

Vital Signs: Standard vital signs (Heart Rate, Blood Pressure, Temperature, and Respiratory Rate) were measured by Emergency Department Personnel or Research Associates in clinically accepted standards using a number of automated devices.

### Statistical Analysis

Data entry and data analysis was performed using IMP 4.0 (SAS Institute, Cary N.C.). After descriptive analyses, standard student t-tests were performed to determine any significant differences between the study groups. Comparisons of hemodynamic and oxygen transport measures were made between two of three study groups (i.e. control vs. SCD baseline, control vs. SCD crisis, and SCD crisis vs. Baseline). The level of significance was set at an alpha of 0.05.

### Results

There were twenty self-reported healthy African-American control subjects, and 61 SCD patients. The median age for the healthy controls was 26±10 years and the median age for the SCD patients was 34±11 years (Table 7).

TABLE 7
Demographics for SCD patients and Healthy Controls

| | Sickle Cell | Healthy | Sickle Cell |
|---|---|---|---|
| | Baseline | Controls | Crisis |
| Age yrs | 33 ± 10 | 26 ± 10 | 36 ± 12 |
| Hb SS | 34 | Normal | 9 |
| Hb SC | 5 | | 6 |
| Hb Sβ-Thal | 5 | | 2 |
| Mean Hgb | 9.3 | 12-14* | 9.45 |
| Gender | 23/21 | 14/6 | 10/7 |
| (M/F) | | | |

*Hgb of 12 for women and 14 for men as low normal standardization

The majority of SCD patients were Hgb SS, and the second most common genotype was Hgb SC (Table 7). The majority of the control subjects were male. There was a nearly even gender distribution in the SCD patients (Table 7). Five of the SCD baseline subjects subsequently were studied as VOC subjects. The sample sizes for these five were too small for further analysis.

Table 8 shows that cardiac hemodynamic profiles (CI, SV, SI) were not statistically significantly different between controls and SCD subjects either at baseline or with VOC (55%±12). There was a trend towards a difference, as shown.

TABLE 8
Comparison of Oxygen Delivery, Oxygen Consumption, Oxygen
Extraction Ratio, and Cutaneous Saturation

| | | P- | | P- | Base- | P- | |
|---|---|---|---|---|---|---|---|
| | Crisis | value | Control | value | line | value | Crisis |
| Cardiac | 5.71 | | 6.12 | | 5.18 | | 5.71 |
| Output | (1.34) | | (1.76) | | (1.48) | | (1.34) |
| l/min | | | | | | | |
| | | .4630 | | .0430 | | .2375 | |
| Cardiac | 3.05 | | 3.24 | | 2.87 | | 3.05 |
| Index | (.56) | | (.69) | | (.68) | | (.56) |
| l/min/m² | | | | | | | |
| | | .4023 | | .0611 | | .3631 | |
| Stroke | 42.5 | | 40.4 | | 41.8 | | 42.5 |
| Index | (10.) | | (9.5) | | (11.6) | | (10.) |
| ml/beat/m² | | | | | | | |
| | | .5477 | | .6560 | | .2375 | |
| Stroke | 78.9 | | 77.51 | | 75.16 | | 78.9 |
| Volume | (22.3) | | (20.4) | | (25.1) | | (22.3) |
| ml/beat | | | | | | | |
| | | .8453 | | .7253 | | .6123 | |
| CtSO2 | 55.2 | | 66.9 | | 57.5 | | 55.2 |
| % | (12.1) | | (8.5) | | (14.4) | | (12.1) |
| | | .0033 | | .0114 | | .6072 | |
| DO2I | 379.3 | | 566.7 | | 368.4 | | 379.3 |
| ml/min/m² | (151.7) | | (121.4) | | (108.1) | | (151.7) |
| | | .0016 | | <.0001 | | .7179 | |
| OERM | .34 | | .25 | | .33 | | .34 |
| % | (.10) | | (.07) | | (.12) | | (.10) |
| | | .0123 | | .0105 | | .5107 | |

Table 8 also shows that DO₂I and SI measurements for healthy control subjects, SCD patients at baseline, and SCD during VOC were different. The DO₂I, in ml O₂/min/m², were 566.7 for control subjects, 368.4 in SCD patients at baseline, and 379.3 for SCD patients in VOC. These differences were statistically significant between healthy control subjects and either SCD patients at baseline or in VOC. They were not statistically significantly different between SCD patients at baseline and SCD patients in VOC.

Table 8 further shows there were statistically differences between groups in tissue oxygenation and extraction. The mean superficial CtSO₂ for control patients was 66.9±8.5%, whereas for vs. SCD patients at baseline it was 57.5±14.4%. A similar significant difference in CtSO₂ was found between control subjects and SCD patients in VOC (CtSO₂.55.2±12.1%). There were similar statistically significant differences in OERM between control and SCD baseline patients, and between control and SCD patients in VOC, whereas there were no OERM differences between SCD baseline patients and SCD patients in VOC.

Last, there were no statistical differences in standard vital sign parameters (Blood Pressure, Heart Rate, Temperature, Respiratory Rate, and SpO₂) between healthy controls and either SCD patients at baseline or SCD patients in VOC.

### Discussion

This study is the first that simultaneously reports both central and tissue level measures of oxygen transport and hemodynamics in SCD patients. The data provide insight that is useful in determining treatments for SCD which may improve oxygen delivery.

Using non-invasive hemodynamic monitoring it was found that SCD patients do not have a significantly different cardiac index, stroke index, heart rate, blood pressure, respiratory rate, or SpO₂ compared to controls. Also, no significant differences were found in these parameters between SCD patients at baseline and those experiencing a VOC. This contrasts to the traditional understanding of SCD as a hyperdynamic, high-output cardiac state, due to the profound anemia that results from the chronic hemolysis of sickled and damaged erythrocytes.

However, the inventors found significant differences between SCD patients at baseline or in VOC and African-American controls in the oxygen transport parameters of DO₂1, CtSO₂, and OER, showing in each case decrements in oxygen transport of SCD patients. Further decrements in oxygen transport were found in comparing SCD patients at baseline to SCD patients in VOC.

Examining potential explanations for the differences in DO₂I, CtSO₂, and OERM between SCD patients (either at baseline or in VOC) and controls, the degree of anemia itself appears to be the mostly likely explanation. Although actual tissue oxygen delivery was not measured, it is not difficult to imagine that a global reduction in DO₂I will result in a decrease in local tissue oxygen delivery, especially to nonessential tissues such as the dermis which was used as the organ monitoring site for CtSO₂. If tissue oxygen consumption does not decrease in the face of decreased tissue oxygen delivery, reductions in venous hemoglobin saturation from a tissue will occur. This happens because either transit time through the tissue is increased, the total available oxygen content in the tissue is reduced, or a combination of both occurs. Thus, it is not surprising that these three values changed together in this study—they are physiologically coupled. And while hemoglobin levels are mathematically coupled with cardiac index in the determination of DO₂I, the measure of CtSO₂ is not dependent on this equation.

What is surprising is that SCD patients do not appear to metabolically compensate for their decreased DO₂ even in their baseline state, despite a lifetime of chronic hemolytic anemia. Such compensation to “normalize” OERM could be envisioned by either tissues reducing their metabolic needs over the long term or by SCD patients having a chronic state of vasodilatation at the microvascular level to improve local tissue oxygen delivery. While it cannot be excluded that either is happening, one can surmise from the findings that compensation is in not enough to normalize CtSO₂ or OERM. The second surprising finding is that SCD patients in the midst of a VOC do not seem to further decompensate from an oxygen transport standpoint. The data indicate that CtSO₂ and OERM may not change because of VOC. Patients in VOC demonstrate a trend to increase their DO₂I likely as a result of an increase in CI. This finding is subject to the limitations discussed below.

Given the data, vasoocclusive sickle cell disease might be viewed as a sub-clinical compensated state of shock as defined by decreases in tissue oxygen delivery on a microcirculatory level (Noguchi, 1993; Ince, 1999; Kumar, 1996; Mentzer, 1980). The introduction of regional measurement techniques has highlighted the inadequacy of the information being garnered by global measurements of oxygenation such as arterial hemoglobin oxygen saturation as well as traditional physical examination findings such as blood pressure, heart rate, and even cardiac output. Therefore, consideration should be given to emphasizing the underlying microcirculation (Krejci, 2000; Zhao, 1985) as reflected in tissue oxygenation as both a diagnostic and therapeutic endpoint.

Using intravital microscopy of the bulbar conjunctiva, Cheung at al. have demonstrated severe microvascular abnormalities in SCD patients both at baseline and during VOC when compared to controls (Cheung, 2002; Cheung, 2001). The abnormalities noted included a combination of reduced microvasularity (loss of capillaries), damaged and distended vessels, reduced red cell velocity, and microvascular sludging. These studies, however, did not examine measures of either central or tissue oxygen transport.

A prior study by has demonstrated decreased RBC flow and tissue hemoglobin oxygen saturation during baseline using visible reference hyperspectral techniques which is also based on differential spectroscopy and blood volume distribution in tissue (Zuzak, 2003). However, this study was performed at baseline and not VOC. In addition, it did not examine parameters of global oxygen delivery simultaneously.

Others performed pulmonary artery catheterization in a group of SCD patients with and without pulmonary hypertension. They found significant decreases in cardiac output and mixed venous hemoglobin oxygen saturation in SCD patients with pulmonary hypertension compared with those without (Anthi, 2007). SCD patients with Pulmonary hypertension also were found to have significantly lower levels of predicted oxygen consumption. However, this study did not perform any local tissue measure of oxygen transport. The degree to which our SCD patients had pulmonary hypertension is unknown but it is interesting to contemplate using CtSO₂ as an index for those that may be at risk or those who should be studied for pulmonary hypertension.

### Conclusion

Sickle cell disease (SCD) is a chronic microcirculatory disease process with frequent acute exacerbations. The vaso-occlusive crisis (VOC) is the most common complication. This process leads to frequent utilization of health care resources and significant impacts to the psychosocial aspects of sickle cell patients. It is documented that sickle cell disease is a complex multifactorial process on a microcirculatory level. The complex interaction of inflammatory cytokines, RBC and RBC interaction, RBC and WBC adhesion, local tissue ischemia, and pain all relate to a microcirculatory dysfunction. In VOC, the final pathway is vascular occlusion mediated by vascular mediators, inflammatory mediators and ischemia. As previously demonstrated in animal models, the vaso-occlusion is reversible and partial in nature. A study by Kaul et. al., that investigated the effects of fluorocarbon emulsion on sickle red blood cell-induced obstruction, found that PFC emulsion treated red cells had a return to baseline oxygenation values (Kumar, 1996). In light of the studies presented hereinabove, and with nitric oxide bioactivity and the beneficial anti-inflammatory and anti-thrombotic effects of PFC make this a novel therapy for SCD. This is an opportunity to obtain better therapies than opiates and fluids during an acute VOC episode.

### Example 4B

A subject having sickle cell disease and suffering from ischemic pain is intravenously or intra-arterially administered an amount of a perfluorocarbon emulsion composition as described herein. The subject experiences reduced or relieved ischemic pain.

### Example 4C

A subject having sickle cell disease and suffering from increased resistance in the peripheral vasculature is intravenously or intra-arterially administered an amount of a perfluorocarbon emulsion composition as described herein. The subject experiences a decrease in peripheral resistance.

### Example 4D

A subject having sickle cell disease and suffering from impaired oxygenation of a tissue is intravenously or intra-arterially administered an amount of a perfluorocarbon emulsion composition as described herein. The administration of the perfluorocarbon or oxygenated perfluorocarbon is effective to increase oxygen delivery to the tissue.

### Example 4E

A subject having sickle cell disease and suffering from an inflamed tissue wherein the inflammation is an effect of the sickle cell disease is intravenously or intra-arterially administered an amount of a perfluorocarbon emulsion composition as described herein. The administration of the perfluorocarbon or oxygenated perfluorocarbon is effective to decrease inflammation of the inflamed tissue.

### Example 4F

A subject suffering a vaso-occlusive crisis is intravenously or intra-arterially administered an amount of a perfluorocarbon emulsion composition as described herein. The administration of perfluorocarbon or oxygenated perfluorocarbon is effective to ameliorate the symptoms of the vaso-occlusive crisis.

### Example 5

### Decompression Sickness

### Example 5A

A subject suffering from decompression sickness is intravenously or intra-arterially administered an amount of a perfluorocarbon emulsion composition as described herein. The administration the PFC emulsion is effective to ameliorate the symptoms of the decompression sickness.

### Example 5B

A subject is intravenously or intra-arterially administered an amount of a perfluorocarbon emulsion composition as described herein prior to being subject to decompression. The administration the PFC emulsion is effective to prevent decompression sickness.

### Example 6

### Air Embolism

### Example 6A

A subject suffering from air embolism is intravenously or intra-arterially administered an amount of a perfluorocarbon emulsion composition as described herein. The administration the PFC emulsion is effective to ameliorate the symptoms of the air embolism.

### Example 6B

A subject suffering from air embolism is intravenously or intra-arterially administered an amount of a perfluorocarbon emulsion composition as described herein. The administration the PFC emulsion is effective to treat the air embolism.

### Example 7

### CNS Trauma Including Traumatic Brain Injury and Spinal Cord Injury

### Example 7A

A subject that has suffered a traumatic brain injury is administered a perfluorocarbon as soon as possible after the injury has occurred. Optionally, the subject is administered a perfluorocarbon emulsion, which can contain oxygen or is saturated with oxygen. Optionally, the subject is administered 50% or 100% oxygen by inhalation. The perfluorocarbon emulsion is Oxycyte® or a similar third-generation perfluorocarbon. The subject has a reduced loss of neuronal tissue as compared to a comparable injured subject who does not receive the perfluorocarbon emulsion.

### Example 7B

A subject that has suffered a traumatic brain injury is administered a perfluorocarbon as soon as possible after the injury has occurred. Optionally, the subject is administered a perfluorocarbon emulsion, which can contain oxygen or is saturated with oxygen. Optionally, the subject is administered 50% or 100% oxygen by inhalation. The perfluorocarbon emulsion is Oxycyte® or a similar third-generation perfluorocarbon. The subject has a reduced ischemic brain damage as compared to a comparable injured subject who does not receive the perfluorocarbon emulsion.

### Example 7C

A subject that has suffered a traumatic brain injury is administered a perfluorocarbon as soon as possible after the injury has occurred. Optionally, the subject is administered a perfluorocarbon emulsion, which can contain oxygen or is saturated with oxygen. Optionally, the subject is administered 50% or 100% oxygen by inhalation. The perfluorocarbon emulsion is Oxycyte® or a similar third-generation perfluorocarbon. The subject has a reduced secondary ischemia as compared to a comparable injured subject who does not receive the perfluorocarbon emulsion.

### Example 7D

A subject that has suffered a traumatic brain injury is administered a perfluorocarbon as soon as possible after the injury has occurred. Optionally, the subject is administered a perfluorocarbon emulsion, which can contain oxygen or is saturated with oxygen. Optionally, the subject is administered 50% or 100% oxygen by inhalation. The perfluorocarbon emulsion is Oxycyte® or a similar third-generation perfluorocarbon. The subject has an increased oxygen tension in a neuronal tissue (brain or spinal cord) as compared to a comparable injured subject who does not receive the perfluorocarbon emulsion.

### Example 8

### Carbon Monoxide Poisoning

A subject suffering from carbon monoxide poisoning is intravenously or intra-arterially administered an amount of a perfluorocarbon emulsion composition as described herein.

The PFC emulsion increases oxygen level in the blood and increases the rate of off-loading of carbon monoxide from hemoglobin in the subject. The administration of the PFC emulsion is effective to treat the carbon monoxide poisoning. Moreover, the perfluorocarbon is well tolerated and has no toxicity.

### Example 9

### Organ Preservation

### Example 9A

A perfluorocarbon emulsion composition as described herein is injected into an organ prior to transplantation.

The PFC emulsion increases oxygen level and oxygen tension in the organ tissue. The organ's survival time period increases. Moreover, the perfluorocarbon is well tolerated and has no toxicity.

### Example 9B

An organ for transplantation is bathed in a perfluorocarbon emulsion composition as described herein prior to transplantation.

The PFC emulsion increases oxygen level and oxygen tension in the organ tissue. The organ's survival time period increases. Moreover, the perfluorocarbon is well tolerated and has no toxicity.

### Example 10

### Wound and Burn Healing and Scar Prevention and Reduction

### Example 10A

A perfluorocarbon emulsion composition as described herein is administered topically to a subject. Specifically, the emulsion is administered topically to a wound on the subject.

The PFC emulsion increases oxygen level and oxygen tension in the wound tissue. In addition, the emulsion accelerates wound healing. Moreover, the perfluorocarbon is well tolerated and has no toxicity.

### Example 10B

A perfluorocarbon emulsion composition as described herein is administered topically to a subject. Specifically, the emulsion is administered topically to a burn wound on the subject.

The PFC emulsion increases oxygen level and oxygen tension in the burnt tissue and surrounding tissue. In addition, the emulsion accelerates the healing of the burn wound. Moreover, the perfluorocarbon is well tolerated and has no toxicity.

### Example 10C

A perfluorocarbon emulsion composition as described herein is administered topically to a subject. Specifically, the emulsion is administered topically to a wound or a scar on the subject.

The PFC emulsion increases oxygen level and oxygen tension in the wound or scarred tissue. In addition, the emulsion accelerates wound healing and ameliorates and reduces the appearance of the scar. Moreover, the perfluorocarbon is well tolerated and has no toxicity.

### Example 11

### Promotion of Anti-Aging

### Example 11A

A perfluorocarbon emulsion composition as described herein is administered topically to a subject. Specifically, the emulsion is administered topically to the skin on the subject.

The PFC emulsion increases oxygen level and oxygen tension in the skin tissue. In addition, the emulsion reduces the appearance of skin imperfection associated with aging including fine lines and wrinkles. Also, the emulsion improves the firmness of the skin where applied. Moreover, the perfluorocarbon is well tolerated and has no toxicity.

### Example 11B

A perfluorocarbon emulsion composition as described herein mixed with caffeine is administered topically to a subject. Specifically, the emulsion mixture is administered topically to the cellulite-affected skin on the subject.

The PFC emulsion mixture increases oxygen level and oxygen tension in the skin tissue. In addition, the emulsion mixture reduces the appearance the cellulite where applied. Moreover, the perfluorocarbon is well tolerated and has no toxicity.

### Example 12

### Treatment of Acne and Rosacea

### Example 12A

A perfluorocarbon emulsion composition as described herein is topically administered to the skin of a subject suffering from acne at the site of the acne. Topical administration of the PFC emulsion is effective to treat the subject's acne. Acne reduction is noticeable, as is a reduction in skin appearance characteristics associated with acne.

### Example 12B

A perfluorocarbon emulsion composition as described herein is topically administered to the skin a subject suffering from acne vulgaris at the site of the acne vulgaris. Topical administration of the PFC emulsion is effective to reduce acne-scarring in the subject by reducing the severity of existing acne vulgaris and preventing or reducing the severity of further acne vulgaris in the subject.

### Example 12C

A perfluorocarbon emulsion composition as described herein is topically administered a subject suffering from a Propionibacterium acnes infection of a skin follicle of the subject. The composition is applied to the skin follicle or the area of skin surrounding the skin follicle. Topical administration of the PFC emulsion is effective to reduce the Propionibacterium acnes infection of the skin follicle of the subject.

### Example 12D

A perfluorocarbon emulsion composition as described herein is topically administered to the skin of a subject suffering from a Propionibacterium acnes infection of the dermis of the subject. The composition is applied to the skin comprising the infected dermis. Topical administration of the PFC emulsion is effective to reduce the Propionibacterium acnes proliferation in the dermis of the subject.

### Example 12E

A perfluorocarbon emulsion composition as described herein is topically administered to the skin of a subject susceptible to acne. Topical administration of the PFC emulsion is effective to prevent or reduce the subject's acne.

### Example 12F

A perfluorocarbon emulsion composition as described herein is topically administered to the skin of a subject wherein there are Propionibacterium acnes in and/or on the skin. Topical administration of the PFC emulsion is effective to kill Propionibacterium acnes in and/or on the skin of the subject.

In the above examples the administration of the composition is one, two or three times per day. The administration can be repeated daily for a period of one, two, three or four weeks, or longer. The administration can be continued for a period of months or years as necessary.

### Example 12G

A perfluorocarbon emulsion composition as described herein is topically administered to the skin of a subject suffering from rosacea at the site of the rosacea. Topical administration of the emulsion composition is effective to treat the subject's rosacea. Rosacea reduction is noticeable, as is a reduction in skin appearance characteristics associated with rosacea.

### Example 13

### Sexual Enhancement

### Example 13A

A perfluorocarbon emulsion composition as described herein is administered topically to sex organs of a human male subject. Local oxygen tension and nocturnal erections are evaluated. Changes in Quality of life (QOL) data is also collected and assessed.

Oxygen level and oxygen tension in the tissue increases. In addition, Quality of life of the subject improves. Moreover, the perfluorocarbon is well tolerated and has no toxicity.

### Example 13B

A perfluorocarbon emulsion composition as described herein is topically administered to sex organs of male and female human subjects. The PFC emulsion is administered once or twice daily. Local oxygen tension and nocturnal erections (in males) are evaluated. Changes in Quality of life (QOL) data is also collected and assessed.

Oxygen level and oxygen tension in the tissue is increases. In addition, Quality of life of the subject improves. Moreover, the perfluorocarbon composition is well tolerated and has no toxicity.

### REFERENCES

1. U.S. Pat. No. 7,445,792 issued Nov. 4, 2008 to Tassu.
2. “Decompression Illness” The Merck Manual, 17^{th} ed. Mark H. Beers, Robert Berkow, eds. Whitehouse Station, N.J.: Merck Research Labs, 1999. pgs. 2465-2467.
3. “Hyperbaric Oxygen Therapy” The Merck Manual, 17^{th} ed. Mark H. Beers, Robert Berkow, eds. Whitehouse Station, N.J.: Merck Research Labs, 1999. pgs. 2497-2503.
4. “Recompression” The Merck Manual, 17^{th} ed. Mark H. Beers, Robert Berkow, eds. Whitehouse Station, N.J.: Merck Research Labs, 1999. pgs. 2467-2468.
5. “Symptoms And Treatment of Specific Poisons” The Merck Manual, 17^{th} ed. Mark H. Beers, Robert Berkow, eds. Whitehouse Station, N.J.: Merck Research Labs, 1999. Table 307-3, pgs. 2623-2644.
6. Adams J H, at al. (1983) “Head Injury in Man and Experimental Animals: Neuropathology.” Atca Neurochir. Suppl., 32:S15-S30.
7. Agarwal G, Wang J C, Kwong S, et al. Sickle hemoglobin fibers: mechanisms of depolymerization. J Mol Biol 2002; 322(2):395-412.
8. Anthi A, Machado R F, Jison M L, et al. Hemodynamic and functional assessment of patients with sickle cell disease and pulmonary hypertension. American journal of respiratory and critical care medicine 2007; 175 (12):1272-9.
9. Bekyarova, G., et al. (1997) “Suppressive effects of FC-43 perluorocarbon emulsion on enhanced oxidative haemolysis in the early postburn phase.” Burns. (23)2: 117-121.
10. Bookchin R M, Lew V L. Pathophysiology of sickle cell anemia. Hematol Oncol Clin North Am 1996; 10(6):1241-53.
11. Bouma, et al. (1992) “Ultra-Early Evaluation of Regional Cerebral Blood Flow in Severely Head Injured Patients Using Xenon Enhanced Computerized Tomography.” J. Neurosurg. 77:360-8.
12. Chen T, Qian Y, Di X, Rice A, Zhu J, Bullock R. Glucose/lactate dynamics after rat fluid percussion brain injury. J Neurotrauma 17(2)135-142, 2000.
13. Cheung A T, Chen P C, Larkin E C, at al. Microvascular abnormalities in sickle cell disease: a computer-assisted intravital microscopy study. Blood 2002; 99(11):3999-4005.
14. Cheung A T, et al. (2001) “Correlation of abnormal intracranial vessel velocity, measured by transcranial Doppler ultrasonography, with abnormal conjunctival vessel velocity, measured by computer-assisted intravital microscopy, in sickle cell disease.” Blood. 97(11):3401-4.
15. Daugherty W P, at al. (May 2004) “Perfluorocarbon Emulsion improves Cerebral Oxygenation and Mitochondrial Function after Fluid Percussion Brain Injury in Rats.” Neurosurgery, 54(5):1223-30; discussion 1230.
16. Davis, Stephen C., et al. (2007) “Topical Oxygen Emulsion: A Novel Wound Therapy” Arch Dermatol. 143(10): 1252-1256.
17. Dehart, R. L.; J. R. Davis (2002). Fundamentals Of Aerospace Medicine: Translating Research Into Clinical Applications, 3rd Rev Ed. United States: Lippincott Williams And Wilkins. pp. 720.
18. Doppenberg E M R, et al. “The Rationale for and Effects of Oxygen Delivery Enhancement to Ischemic Brain in a Feline Model of Human Stroke.” An NY Acad. Sciences, 825:241-257.
19. Doppenberg, E, Watson, J., et al. Intraoperative monitoring of substrate delivery during aneurysm and hematoma surgery: initial experience in 16 patients. J. Neurosurg, 1997, 87:809-816.
20. Eady et al., (1989) “Erythromycin resistant propionibacteria in antibiotic treated acne patients: Association with therapeutic failure” Br J Dermatol. 1989 July; 121(1):51-7.
21. Evans, et al. (1987) “Membrane-associated sickle hemoglobin: a major determinant of sickle erythrocyte rigidity.” Blood. 70(5):1443-9.
22. Fabry M E, Nagel R L. The effect of deoxygenation on red cell density: significance for the pathophysiology of sickle cell anemia. Blood 1982; 60(6):1370-7.
23. Fixler J, Styles L. Sickle cell disease. Pediatr Clin North Am 2002; 49(6):1193-210, vi.
24. Garrison, et al. (1998) “Microvascular changes explain the “two-hit” theory of multiple organ failure.” Ann Surg 227(6):851-60.
25. Guyton A, ed. The Systemic circulation: In Textbook of Medical Phsiology. 6th ed. Philadelphia: W.B. Saunders; 1981.
26. Hogan, at al. (2007) “Peripheral Tissue Oxygenation Extraction Abnormalities Persist in Acutely Decompensated Heart Failure After Emergency Department Treatment. Acad Emerg Med 2007(S):116.
27. Ince C, Sinaasappel M. Microcirculatory oxygenation and shunting in sepsis and shock. Crit Care Med 1999; 27(7):1369-77.
28. Ingram V M. A specific chemical difference between the globins of normal human and sickle-cell anaemia haemoglobin. Nature 1956; 178(4537):792-4.
29. Jennett, B. (2005) “Development of Glasgow Coma and Outcome Scales” Nepal Journal of Neuroscience, 2:24-28.
30. Kaneda, at al. (2009) “Perfluorocarbon nanoemulsions for quantitative molecular imaging and targeted therapeutics” Ann Biomed Eng. 37(10) October 2009. NDN 230-1024-9131-6.
31. Kaul D K, Hebbel R P. Hypoxia/reoxygenation causes inflammatory response in transgenic sickle mice but not in normal mice. J Clin Invest 2000; 106(3):411-20.
32. Krejci V, at al. (2000) “Continuous measurements of microcirculatory blood flow in gastrointestinal organs during acute haemorrhage.” Br J Anaesth. 84(4):468-75.
33. Kumar A, at al. (1996) “Phorbol ester stimulation increases sickle erythrocyte adherence to endothelium: a novel pathway involving alpha 4 beta 1 integrin receptors on sickle reticulocytes and fibronectin.” Blood 88(11):4348-58.
34. Kwon, et al. (2005) “Effect of perfluorocarbons on brain oxygenation and ischemic damage in an acute subdural hematoma model in rats.” J Neurosurg. October:724-730, 2005.
35. Leach, et al. (1998) “ABC of Oxygen—Hyperbaric Oxygen Therapy” British Medical Journal—Clinical Review. 317:1140-1143.
36. Levin, S. at al., (2001) “Validity and Sensitivity to Change of the Extended Glasgow Outcome Scale in Mild to Moderate Traumatic Brain Injury” Journal of Neurotrauma. June 2001, 18(6): 575-584.
37. Leviton and Pallansch (1959) J. Dairy Science, 42(1):20-27.
38. Lifshitz, at al. (2004) “Mitochondrial damage and dysfunction in traumatic brain injury.” Mitochondrion (5-6)705-713.
39. Mason, R P et al. (1989) “Perfluorocarbon imaging in vivo: a 19F MRI study in tumor-bearing mice” Magn Reson Imaging. Vol. 7 Issue 5 Pg. 475-85.
40. Mentzer W C, Jr., Wang W C. Sickle-cell disease: pathophysiology and diagnosis. Pediatr Ann 1980; 9(8):287-96.
41. Menzel, et al. (1999) “Increased Inspired Oxygen Concentration Improves Brain Tissue Oxygenation and Tissue Lactate Levels after Severe Human Head Injury.” J. Neurosurg. 91(1):1-10.
42. Noguchi C T, Schechter A N, Rodgers G P. Sickle cell disease pathophysiology. Baillieres Clin Haematol 1993:6(1):57-91.
43. Nortje at al.: Effect of hyperoxia on regional oxygenation and metabolism after severe traumatic brain injury: Preliminary findings. Crit Care Med 36:273-281, 2008.
44. Nortje J, Gupta A K. The role of tissue oxygen monitoring in patients with acute brain injury. British Journal of Anesthesia, 2006, 97(1):95-106.
45. Pennock B E. Is measurement of cardiac output using impedance cardiography accurate? Chest 1997; 111(6):1786.
46. Prockop L D, Chichkova R I (November 2007). “Carbon monoxide intoxication: an updated review”. Journal of the Neurological Sciences 262 (1-2): 122-130
47. Reinert M, et al. (2000) “High levels of extracellular potassium and its correlates after severe head injury: Relationship to high ICP.” J Neurosurg 93:810-817.
48. Robertson C. Personal communication, 2004.
49. Shen, Yao, et al. (2007) “Carnosine attenuates mast cell degranulation and histamine release induced by oxygen-glucose deprivation” Cell Biochemistry and Function. 26(3):334-338.
50. Silver, J., et al. (2005) “Neural Pathology” Textbook Of Traumatic Brain Injury. Washington, D.C.: American Psychiatric Association. Chap. 2, pp. 27-33.
51. Spahn, D R (1999) “Blood Substutes—Artificial Oxygen Carriers: Perfluorocarbon Emulsion” Cirt Care. 3:R93-R97.
52. Spiess, B D (2009) “Perfluorocarbon emulsions as a promising technology: a review of tissue and vascular gas dynamics.” J Appl Physiol. 106: 1444-1452.
53. Stiefel M F, et al. Reduced mortality rate in patients with severe traumatic brain injury treated with brain tissue oxygen monitoring. J Neurosurg 2005 November; 103(5):805-811.
54. Tavalin S J, Ellis E F, Satin L S. Mechanical perturbation of cultured cortical neurons reveals stretch induced delayed depolarization. J Neurophysiol. 74, 2767-2773, 1995.
55. Thiboutot at al., (1997) “Acne. An overview of clinical research findings” Dermatol Clin. 1997 January; 15(1):97-109.
56. Tobias C, Reinert M, Seiler R, Gilman C, Scharf A, Bullock R. Normobaric hyperoxia induced improvement in cerebral metabolism and reduction in intracranial pressure in patients with severe head injury: a prospective cohort matched study. J. Neurosurg. 101:435-444, 2004.
57. Tobin M J, ed. Principles and Practice of Intensive Care Monitoring. New York: McGraw-Hill; 1998.
58. U.S. Navy Supervisor of Diving (2008). “Chapter 20: Diagnosis and Treatment of Decompression Sickness and Arterial Gas Embolism” (PDF). U.S. Navy Diving Manual. SS521-AG-PRO-010, revision 6. volume 5. U.S. Naval Sea Systems Command. p. 37.
59. Valadka A. Gopinath S P, Contant C F, Uzura M, Robertson C S. Relationship of Brain Tissue PO2 to Outcome After Severe Head Injury. Crit. Care Med., 1998, 26:1576-1581.
60. Van De Water J M, et al. (2003) “Impedance cardiography: the next vital sign technology?” Chest. 123(6):2028-33.
61. Vann, R D (1989) “The Physiological Basis of Decompression.” 38th Undersea and Hyperbaric Medical Society Workshop. UHMS Publication Number 75(Phys)6-1-89.: 437.
62. Verweij B, Muizelaar P, Vinas F, Patterson P, Xiong Y, Lee C P. Impaired cerebral mitochondrial function after traumatic brain injury in humans. J Neurosurg 93(5):815-820; 2000.
63. Ward K R, Ivatury R R, Barbee R W, et al. Near infrared spectroscopy for evaluation of the trauma patient: a technology review. Resuscitation 2006; 68(1):27-44.
64. Wilson, et al. (1998) “Structured Interviews for the Glasgow Outcome Scale: Guidelines for Their Use” J. Neurotrauma, 15(8):573-585.
65. Wolff K D, Kolberg A, Mansmann U. Cutaneous hemoglobin oxygenation of different free flap donor sites. Plast Reconstr Surg 1998; 102(5):1537-43.
66. Wolff K D, Marks C, Uekermann B, Specht M, Frank K H. Monitoring of flaps by measurement of intracapillary haemoglobin oxygenation with EMPHO II: experimental and clinical study. Br J Oral Maxillofac Surg 1996; 34(6):524-9.
67. Zauner A, Bullock R, Di X, Young H F. Brain Oxygen, CO2, pH, and Temperature Monitoring: Evaluation in the Feline Brain. Neurosurgery, 1995, 37:1167-1177.
68. Zauner A, Bullock R, Young H F. Continuous Brain Oxygen, CO2, pH and Temperature Monitoring in Neurosurgical Patients. Neurosurgery, 1995, 37:570-575.
69. Zauner A, et al. (1997) “Continuous monitoring of cerebral substrate delivery and clearance: initial experience in 24 patients with severe acute brain injuries.” Neurosurgery 41:1082-1091; discussion 1091-1083.
70. Zauner A, at al. (1997) “Multiparametric continuous monitoring of brain metabolism and substrate delivery in neurosurgical patients.” Neurol Res 19:265-273.
71. Zhao K S, at al. (1985) “Microvascular adjustments during irreversible hemorrhagic shock in rat skeletal muscle.” Microvasc Res. 30(2):143-53.
72. Zhou A, at al. (2008) “Perfluorocarbon emulsion improves cognitive recovery following fluid percussion brain injury in rats. Neurosurgery. 63:799-807”.
73. Zuzak K J, at al. (2003) “Imaging hemoglobin oxygen saturation in sickle cell disease patients using noninvasive visible reflectance hyperspectral techniques: effects of nitric oxide.” Am J Physiol Heart Circ Physiol. 285(3):H1183-9.

What is claimed is:

1-21. (canceled)

22. A method of manufacturing a perfluorocarbon emulsion comprising the steps:
a) mixing an emulsifier and aqueous medium together;
b) adding perfluorocarbon to the mixture of step a);
c) mixing the mixture of step b) to form a coarse emulsion;
d) obtaining a sample of the coarse emulsion of step c) and determining particle size distribution of the sample;
e) if the sample of step d) has a monomodal particle size distribution, then homogenizing the coarse emulsion of step c); and
f) obtaining the emulsion.

23. The method of claim 22, wherein in step e) the coarse emulsion of step c) is homogenized only if the median particle size of the sample of step d) is less than 20 μm.

24. The method of claim 22, wherein in step e) the coarse emulsion is homogenized at or above 7,000 psi.

25. A process for preparing a pharmaceutical product containing a PFC emulsion, the process comprising:
a) obtaining a batch of perfluorocarbon emulsion or coarse emulsion;
b)1) determining the particle size distribution of the batch;
2) determining the total amount of residual fluoride present in the batch; or
3) determining the total amount of lysophosphatidylcholine (LPTC) present in the batch; and
c) preparing the pharmaceutical product from the batch only if 1) the batch is determined to have a monomodal particle size distribution; 2) the batch is determined to have less than 40 ppm residual fluoride by weight of the emulsion; or 3) the batch is determined to have less than 7 g/L lysophosphatidylcholine (LPTC) by weight of the emulsion.

26. A process for validating a batch of an emulsion for pharmaceutical use, the process comprising:
a)1) determining the particle size distribution of a sample of the batch;
2) determining the total amount of residual fluoride in a sample of the batch; or
3) determining the total amount of lysophosphatidylcholine (LPTC) in a sample of the batch; and
b) validating the batch for pharmaceutical use only if 1) the sample of the batch has a monomodal particle size distribution; 2) the batch contains less than 40 ppm residual fluoride by weight of the emulsion; or 3) the batch contains less than 7 g/L lysophosphatidylcholine (LPTC) by weight of the emulsion.

27. The process of claims 26, wherein in steps a)1)-a)3) are performed after the sample of the batch has been subjected to stability testing.

28. A pharmaceutical product containing a PFC emulsion prepared by the process of claim 25.

29. A method of treating sickle cell disease, decompression sickness, air embolism or carbon monoxide poisoning in a subject suffering therefrom comprising administering to the subject the pharmaceutical product of claim 28 effective to treat the subject's sickle cell disease, decompression sickness, air embolism or carbon monoxide poisoning.

30. A method of preserving an organ prior to transplant comprising contacting the organ with the pharmaceutical product of claim 28 effective to increase the organ's survival time.

31. A method of treating a wound, a burn injury, acne or rosacea in a subject suffering therefrom comprising topically administering to the skin of the subject the pharmaceutical product of claim 28 effective to treat the subject's wound, burn injury, acne or rosacea.

32. A method of increasing the firmness of the skin or reducing the appearance of fine lines, wrinkles or scars in a subject comprising topically administering to the skin of the subject the pharmaceutical product of claim 28 effective to increase the firmness of the subject's skin or reduce the appearance of fine lines, wrinkles or scars on the subject's skin.
